(19) United States
(12) Patent Application Publication
(10) Pub. No.: US 2023/0192766 A1
(43) Pub. Date: Jun. 22, 2023

(54) ADAPTIVE RECOMBINANT NANOWORMS FROM GENETICALLY ENCODABLE STAR AMPHIPHILES

(71) Applicants: Davoud Mozhdehi, Syracuse, NY (US); Md Shahadat Hossain, Syracuse, NY (US)

(72) Inventors: Davoud Mozhdehi, Syracuse, NY (US); Md Shahadat Hossain, Syracuse, NY (US)

(73) Assignee: SYRACUSE UNIVERSITY, SYRACUSE, NY (US)

(21) Appl. No.: 18/053,517

(22) Filed: Nov. 8, 2022

Related U.S. Application Data
(60) Provisional application No. 63/276,943, filed on Nov. 8, 2021.

(51) Int. Cl. C07K 7/06; C12N 15/63

(52) U.S. Cl. CPC C07K 7/06; C12N 15/63

(57) ABSTRACT

A programmable assembly of proteins into well-defined nanoworms with broadened stability regimes is disclosed. Posttranslational modifications (PTMs) were used to generate lipidated proteins with precise topological and compositional asymmetry. Using an integrated experimental and computational approach, the material properties (thermoresponse and nanoscale assembly) of these hybrid amphiphiles are modulated by their amphiphilic architecture. The judicious choice of amphiphilic architecture can be used to program the assembly of proteins into adaptive nanoworms that undergo a morphological transition (sphere-to-nanoworms) in response to temperature stimuli.

### CROSS-REFERENCE TO RELATED APPLICATIONS

This application claims priority to U.S. Provisional Application No. 63/276,943 filed on Nov. 8, 2021.

### STATEMENT REGARDING FEDERALLY SPONSORED RESEARCH AND DEVELOPMENT

This invention was made with government support under Grant No. 1R35GM142899-01 awarded by the National Institutes of Health (NIH) and Grant No. 2105193 awarded by the National Science Foundation. The government has certain rights in the invention.

### INCORPORATION BY REFERENCE

The Sequence Listing XML file submitted via the USPTO patent electronic filing system named 156P656fromWIPOsoftware.xml, created on Nov. 8, 2022, and having a size of 10 kilobytes is hereby incorporated by reference.

### BACKGROUND OF THE INVENTION

### 1. Field of the Invention

The present invention relates to nano-encapsulation materials, and more specifically, to star-shaped amphiphilic fatty acid-modified elastin-like polypeptides constructs.

### 2. Description of the Related Art

Nano-encapsulation of therapeutics and imaging agents can dramatically improve their efficacy and specificity, while reducing their undesirable side-effects. However, as the use of nanomaterials in medicine expands, new concerns regarding their off-target accumulation and toxicity have emerged. Nanobiomaterials, such as proteins, are promising platforms to address these concerns because in addition to degradability their sequence, structure, and function can be controlled with precision to modulate the carriers' characteristics such as targeting, stealth, and immunomodulation, among others. Consequently, precise engineering of the size and morphology of protein-based nanomaterials remains a key objective of the field as these characteristics regulate the pharmacokinetics and biodistribution of the encapsulated cargo. Specifically, rods are receiving increased attention because the higher aspect ratios of these anisotropic nanoparticles can increase cellular internalization and interaction with cell-surface receptors. Despite these promising attributes, the molecular design rules to create protein-based rods with both radius and length below 200 nm (also known as nanoworms) remain unclear.

The rational design of nanoworms requires delicate optimization of building blocks' “conformational asymmetry,” because these assemblies are thermodynamically favorable only in a narrow range of the phase diagram. The conformational asymmetry of macromolecules can be adjusted by altering their amphiphilic composition and/or topology. However, because proteins are only expressed as a linear sequence of amino acids, the design of protein-based nanorods has exclusively relied on constructs with extreme compositional asymmetry. For instance, some researchers have designed nanoworms (NWs) by fusing large, disordered elastin-like polypeptides (ELPs) to short dissimilar domains such as single-chain variable domain fragments or aromatic peptides. However, the complex and nonintuitive dependence of the NW's properties on protein sequence and features limits the widespread utility of this linear amphiphilic architecture. This is because small perturbations in composition or changes to solution parameters can result in polydisperse mixtures of cylindrical assemblies whose lengths range from nano- to micrometer. These difficulties in synthesis may hinder applications such as drug delivery or templated synthesis of nanomaterials, in which dispersity alters performance metrics such as biodistribution, endocytosis, and other desired functions of nanomaterials. Accordingly, there is a need in the art for a new class of protein-based nanostructures for biomedical applications using topological engineering of proteins to facilitate access to unique assemblies such as nanoworms by modulating their stability boundaries.

### BRIEF SUMMARY OF THE INVENTION

The present invention comprises the molecular design of star-shaped amphiphilic fatty acid-modified elastin-like (SAFE) polypeptide constructs. Examples demonstrate that their material properties (assembly and thermoresponse) are modulated by their lipidation pattern as characterized by scattering and microscopy experiments. Using molecular dynamics simulations and principal component analysis, we reveal that the lipidation pattern influences the shape, size, and hydration of SAFE chains at the molecular level and that the changes in these microscopic features parallel observed trends in macroscopic properties as a function of lipidation pattern.

Examples of the present invention focused on the simplest nonlinear topology: the miktoarm star in which two hydrophobic arms are compositionally identical while the third hydrophilic arm differs, i.e., A₂B. To manipulate the protein's topology (e.g., branching), the isopeptide ligation between split-protein pairs, SpyCatcher and SpyTag, was used. This strategy has been used to synthesize proteins with complex nonlinear topologies with enhanced stability and proteolytic resistance or to click bio-active motifs to protein nanostructures. However, controlling nano-assembly of proteins by topological engineering alone remains limited because it may not provide the energetic driving force to compensate for the entropic penalty of self-organization. To overcome this barrier and induce nano-assembly, topological engineering was combined with lipidation PTM to generate hybrid protein amphiphiles with topological and compositional asymmetry.

In the present invention, the arms of the star (A or B) are based on a model thermoresponsive ELP with the canonical sequence of (GXGVP)ₙ whose composition arm is distinguished by the identity of the guest residue (X) and arm (n) length. Together these features determine the arm's interaction with water or with each other. The N-termini of the hydrophobic arms were modified with a myristoyl group (C14:0) to generate star-shaped amphiphilic fatty acid-modified elastin-like polypeptides (SAFE). The amphiphilic architecture of SAFEs is defined by the hierarchical combination of the star topological asymmetry (compositional differences between the arms) and the pattern of lipidation (i.e., number and location). The inter- and intra-arm interactions and the hydration of the arms could be modulated by changing the pattern of lipidation and/or the solution temperature, thus providing a dial to regulate the nano-assembly of SAFEs into NWs.

In a first aspect, the present invention is a star miktoarm formed from a first hydrophobic arm comprised of a first repeating peptide unit having a first C-terminus and a first N-terminus, a first hydrophilic arm comprised of a second repeating peptide unit having a second C-terminus and a second N-terminus, wherein the first hydrophilic arm is bound to the first hydrophobic arm at a junction formed by the second N-terminus and the first C-terminus, and a second hydrophobic arm comprised of a third repeating peptide unit having a third C-terminus and a third N-terminus, wherein the second hydrophobic arm is bound by the third C-terminus to the junction of the first hydrophilic arm and the first hydrophilic arm. The first repeating peptide unit and the third repeating peptide unit may be the same. At least one of the first hydrophobic arm and the second hydrophobic arm may be myristoylated and, in some cases, both the first hydrophobic arm and the second hydrophobic arm may be myristoylated. The junction is formed by a first peptide fusion protein, such as SpyTag. The second hydrophobic arm is bound to a second peptide fusion protein, SpyCatcher, that will irreversibly conjugate with the first peptide fusion protein.

In another aspect, the present invention is a method of making a star miktoarm, comprising the steps of forming a first hydrophobic arm comprised of a first repeating peptide unit having a first C-terminus and a first N-terminus, forming a first hydrophilic arm comprised of a second repeating peptide unit having a second C-terminus and a second N-terminus, binding the first hydrophilic arm to the first hydrophobic arm at a junction formed by the second N-terminus and the first C-terminus, forming a second hydrophobic arm comprised of a third repeating peptide unit having a third C-terminus and a third N-terminus, and then binding the second hydrophobic arm by the third C-terminus to the junction of the first hydrophilic arm and the first hydrophilic arm.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a series of schematics showing the synthesis and nomenclature of miktoarm star amphiphiles: a) The architecture of plasmids used for the synthesis of SAFE's linear building blocks. Two pETDuet-1 plasmids were used to encode all genetic elements necessary for biosynthesis of SAFEs including ELP arms, N-myristoyltransferase (NMT) and bipartite SpyTag/Catcher proteins for lipidation and branching PTMs; b) A schematic of the reaction between two model linear building blocks to generate a representative miktoarm star; c) The identity of ELP's guest residue (i.e., hydrophobic valine or hydrophilic serine) and the lipidation pattern of the hydrophobic arms define the amphiphilic architecture of each construct. SAFE constructs are labelled using a three-letter code based on identity of the functional group terminating each arm. “N” and “M” refer to the free amine (unmodified) or myristoyl (modified) hydrophobic arms, and C corresponds to the carboxylic acid of hydrophilic arm. The first two letters refer to the hydrophobic arms that are linearly fused to serine block or catcher domain, respectively. NNC—non-lipidated, MNC and NMC—single-lipid, and MA/IC—double-lipid amphiphiles.

FIG. 2 is a series of images of monitoring isopeptide formation reactions of unmodified and myristoylated linear building blocks using SDS-PAGE. In each panel, lanes 1 and 2 are starting materials—the ELP diblock copolymer, (M)-(GVGVP)₄₀(SEQ ID NO: 1)-Tag-(GSGVP)₆₀(SEQ ID NO: 2) and ELP fused to Catcher, (M)-(GVGVP)₄₀(SEQ ID NO: 1)-Catcher. Lanes 3 and 4 are reaction mixtures at time 0 and 2 h. Myristoylation did not alter the reactivity of Spy pairs under these conditions.

FIG. 3 is a series of graphs showing Molecular characterization of purified linear building blocks (and controls): (a) Analytical RP-HPLC of each component. Modification with the hydrophobic myristoyl group increased the retention time of the protein. V₄₀-Tag and M-V₄₀-Tag were analyzed on C4 columns because of their high hydrophobicity. All other constructs were analyzed using a C18 column; and (b) MALDI-TOF-MS analysis of proteins. Modification with myristic acid (and removal of water) increased the m/z ratio by 210. The vertical dotted line in b denotes the theoretical (calculated) molecular weight of each construct.

FIG. 4 is a schematic showing SAFE amphiphiles produced using a one-pot recombinant expression, tandem PTM process: (a) A schematic of the proof-of-concept experiment using orthogonal plasmids (with compatible origins of replication and antibiotic selection markers) for co-expression of NMT, V40-Tag-S60, and V40-Catcher proteins in one cell. After cell lysis, phase separation of all proteins fused to ELP domains was triggered by the addition of kosmotropic salts at 40° C. The protein pellet was separated from the supernatant and redissolved in water:ethanol mixture (1:1 v/v) for analysis using SDS-PAGE and MALDI; (b) The presence of both SpyCatcher and SpyTag are required for branching PTM (cf. lane 3 with lanes 1 and 2; and (c) MALDI-TOF-MS was used to confirm the molecular weight of MMC produced in the one-pot reaction.

FIG. 5 depicts the purity of SAFE amphiphiles as confirmed by SDS-PAGE (a) and analytical reverse-phase HPLC (b).

FIG. 6 is a graph of the N-terminal myristoylation was confirmed by digestion of the proteins with trypsin and the analysis of peptide fragments using MALDI-TOF-MS. The N-terminal glycines of V₄₀-Tag-S₆₀ (top) or V₄₀-Catcher expressed in the presence of NMT were myristoylated.

FIG. 7 is a series of graphs showing a MALDI-TOF-MS analysis of SAFE amphiphiles: (a) NNC, (b) MNC, (c) NMC, and (d) MMC. The vertical dashed and dotted lines are drawn to denote the theoretical (calculated) [M+H]⁺ and [M+2H]²⁺, respectively.

FIG. 8 is a series of graphs showing that lipidation patterns modulate the thermoresponse of miktoarm star amphiphiles: a) Turbidity profiles of SAFE amphiphiles at 20 μM in PBS as a function of temperature; b) The concentration dependence of the SAFE's transition temperatures. The shaded area in FIG. 8b represents a 90% confidence interval for the fitted line. The horizontal line at 0.15 in FIG. 8a is drawn to schematically distinguish between transition temperatures resulting in highly turbid solutions (NMC and MNC) vs. transitions that only partially increased solution concentration (NNC and MMC). The Tₜ of NNC/MMC exhibited lower concentration dependence (shallower slope) than single-lipid amphiphiles. The thermal behavior of single-lipid amphiphiles showed subtle differences based on which hydrophobic arm was lipidated.

FIG. 9 is a series of graphs of the turbidity profiles of star amphiphiles at different concentrations (5-30 μM in PBS). The inset depicts the evolution of the turbidity profile close to the observed transition temperatures. The turbidity of both NNC and MMC solutions only increased modestly with temperature (a, d), while the turbidity of single-lipidated MNC and NMC solutions (b and c), increased significantly above transition temperature. The phase-behavior of single-lipidated constructs varied significantly with their concentration.

FIG. 10 is a series of graphs of the characterization of thermoresponse (and its concentration dependence) for linear controls in PBS using turbidimetry: (a) V₄₀-Tag and M-V₄₀-Tag; (b) V₄₀-Tag-S₆₀ and M-V₄₀-Tag-S₆₀; (c) V₄₀-Catcher and M-V₄₀-catcher; (d) The concentration dependence of linear constructs transition temperatures. In all panels, nonmyristoylated samples are represented with open symbols (and dashed lines), while lipidated controls are shown using filled symbols (and solid lines).

FIG. 11 is a series of graphs showing that dynamic light scattering confirms that lipidation pattern modulates the temperature-dependent assembly of star amphiphiles. (a) Autocorrelation functions of each construct dissolved in PBS (20 μM) at 20° C. (blue solid line), 40° C. (purple dashed line), and 60° C. (red dotted line). (b) A bubble plot summarizing the size of aggregates derived from the cumulant method. The center of each circle represents the average hydrodynamic radius (Z_{avg}), while the area of the bubble represents the polydispersity index (PDI) at each temperature. NNC only formed small assemblies when heated above 50° C., while all lipidated samples assembled even below their Tₜ. The size of lipidated SAFE assemblies increased with temperature, albeit a divergent behavior was observed depending on their lipidation patterns. NMC mostly transitioned into large mesoscale aggregates at temperatures above 40° C., while MNC formed a mixture of small and large assemblies. The increase in PDI as a function of temperature is consistent with the formation of a mixture of assemblies with different sizes or characteristics. The size of MMC assemblies initially increased with temperature but remained unchanged above 30° C. with a low PDI (<0.1). Error bars are standard deviations of three measurements. Lines are added as a visual reference.

FIG. 12 is a series of images showing the microscopic characterization of lipidated SAFE's assembly at nano-/mesoscale: (a-c) MNC; (d-f) NMC; and (g-i) MMC. MNC forms a mixture of spherical and elongated aggregates at 20° C., and high-aspect-ratio bottle brushes with a well-defined diameter (75±20 nm), but polydisperse lengths (261±172 nm) at 40° C. NMC forms spherical assemblies at 20° C., and nano-tapes at 40° C. Compared to MNC bottlebrushes, the core of these structures (visualized as white areas) was wider, but their corona was less resolved. In contrast, MMC formed a mixture of spherical and elongated nanoworms at 20° C. The spherical assemblies were converted to nanoworms at 40° C. with a well-defined size. At 60° C., both single-lipid constructs undergo liquid-liquid phase separation and form micron-size coacervates (consistent with turbidimetry and DLS data). However, MMC nanoworms aggregates were stable at high temperatures, and no bulk-phase separation was detected in DIC.

FIG. 13 is a series of representative TEM images for linear (a-h) and star amphiphiles (i-l), and the select statistical size distributions derived from image analysis (m-o). (a) M-V₄₀-Tag formed long fibers at 40° C. M-V₄₀-Tag-S₆₀ formed spherical micelles at 20° C. (b), and a polydisperse mixture of worm-like micelles at 40° C. (c, d). M-V₄₀-Catcher formed a polydisperse mixture of worm-like micelles at 20° C. (e, f) and 40° C. (g, h). NNC formed spherical micelles at 60° C. (i). Both MNC and NMC formed worm-like micelles at 40° C. (j, k), but the morphology of these micelles differs based on the location of the attached lipid. MNC forms polydisperse worm-like materials (WLMs) with canonical cylindrical morphology. However, NMC forms shorter micelles with noticeably larger cores (visualized as the white area in the stained images). Both constructs formed coacervates at elevated temperatures. (l) MMC formed stable nanoworms with narrow polydispersity at 40° C. (m, n) The size-distribution histograms for the diameter of spherical particles formed by M-V₄₀-Tag-S₆₀ (at 20° C.) and NNC (at 60° C.). Measurement results are reported as mean±SD. (o) A violin plot showing the length-distribution of constructs that formed anisotropic worm-like micelles. Unless specified, the sample temperature is 40° C. The horizontal dashed line denotes the median value.

FIG. 14 is a series of graphs of the schematic phase diagram for temperature-dependent nano-assembly of linear controls (a) and star amphiphiles (b) derived from the collection of turbidimetry, DLS, and TEM studies. The dashed black vertical lines show the approximate temperature range for concentration-dependent transitions in nano- or meso-assemblies. The solid vertical lines are added to denote concentration-independent transitions. The combination of dual lipidation and branching in MMC is necessary to form nanoworms over a broad window of temperature and concentration ranges. WLM is worm-like micelles.

FIG. 15 is a series of trellis plots depicting the change in 15 molecular features as a function of simulation time at 5° C. (a-c) and 67° C. (d-f) for star amphiphiles. (a,d) The pair-wise distance between the ELP arms in angstroms. (b,e) The radii of gyration of different domains. (c,f) The average number of water molecules in the first hydration layer and the number of hydrogen bonds between water and residues in each domain. VC: The V₄₀ linearly fused to the Catcher domain; VT: V₄₀ linearly fused to the Tag domain; S: S₆₀. Catcher-Tag refers to the branching point formed after the reaction between SpyCatcher and SpyTag. Simulation data are plotted at 1 ns interval between 170-200 ns.

FIG. 16 is a series of depictions showing that the lipidation pattern alters the physicochemical properties of star amphiphiles at the single-chain level. a) Atomistic conformations of NNC, MMC, MNC, and NMC structures (front and back, cartoon representation) along with their first hydration shell (dots) at 37° C. Color scheme for the structures: SpyCatcher (light blue), SpyTag (red), V₄₀ fused to SpyCatcher (cyan), V₄₀ fused to SpyTag (teal), S₆₀ (orange). The attached lipids are shown as spheres and colored based on the color of the attached ELP. b) Principal component analysis enables the clustering of MD simulation results into largely nonoverlapping clusters. PC axis 1 correlates with temperature changes while PC2 discriminates single-lipid amphiphiles from symmetrically non- or double-lipidated NNC and MMC. PC3 captures the variations between the single-lipid constructs MNC and NMC. In both panels, the open symbols and dashed lines refer to simulations at 5° C., while filled symbols refer to results at 67° C. NNC (circle), MMC (diamond), MNC (square), and NMC (triangle). c) The heat map depicts the contribution (loading) of each molecular feature to PC1-3, with blue and red representing negative or positive loadings. The lipidation pattern and temperature modulate size, shape (form), and hydration of star amphiphiles. F1-3 represent the pair-wise distance between different arms; S1-S4 represent the size of each arm and the branching point. H1-H8 represent the number of water molecules in the hydration shell and the number of hydrogen bonds between the solvent and each domain. See methods for the definition of each variable.

FIG. 17 is a series of graphs showing component selection using parallel analysis (a) and proportion of variance contained within each principal component (PC) (b). The first 3 PCs account for ˜75% of variations in the dataset.

FIG. 18 is a table of DLS results derived from the analysis of autocorrelation functions using cumulants methods, where ^{a}—Average hydrodynamic diameter (nm) derived from the cumulants method (n=3); ^{b}—Polydispersity index derived from the cumulants method, (n=3); n.d.—not determined.

FIG. 19 is a schematic of cloning steps used to construct plasmids to produce SAFE amphiphiles and the linear controls used. The genes encoding the main building blocks (V₄₀, S₆₀, SpyCatcher, and SpyTag) were cloned into pET-24a(+) plasmids. Recursive directional ligation was used to assemble the genes for fusion proteins in the desired order. The assembled gene was then subcloned into pETDuet-1 vectors. These bicistronic vectors were used to co-express the NMT enzyme and the desired protein fused to NMT peptide substrate to produce myristoylated proteins in E. coli.

FIG. 20 is a series of graphs of dynamic light scattering analysis of the temperature-dependent assembly of linear controls. A bubble plot summarizing the size of aggregates derived from the cumulant method, (a) V₄₀-Tag and M-V₄₀-Tag; (b) V₄₀-Tag-S₆₀ and M-V₄₀-Tag-S₆₀; (c) V₄₀-Catcher and M-V₄₀-Catcher; and (d) superimposition of data presented in a-c. The symbol in the center of each bubble represents the average hydrodynamic radius (Z_{avg}), while the area represents the polydispersity index (PDI). In all panels, nonmyristoylated samples are represented with open symbols (and dashed lines), while lipidated controls are shown using filled symbols (and solid lines). Lines are added as a visual reference. All proteins were analyzed at 20 μM in PBS, except M-V₄₀-Tag (30 μM in PBS). Error bars are standard deviations of three measurements.

FIG. 21 is a series of graphs of the intensity distributions for star amphiphiles at 20° C. (bottom), 40° C. (middle panel), and 60° C. (top panel) derived from the analysis of ACFs shown in FIG. 11a with the CONTIN algorithm. All proteins were analyzed at 20 μM in PBS. Error bars are standard deviation of three measurements.

FIG. 22 is a series of graphs of the intensity distributions for linear unmodified (dashed bars) and myristoylated (solid bars) control amphiphiles at 20° C. (bottom), 40° C. (middle panel), and 60° C. (top panel) derived from the analysis of CRFs with the CONTIN algorithm. (a,b) V₄₀-Tag and M-V₄₀-Tag; (c, d) V₄₀-Tag-S₆₀ and M-V₄₀-Tag-S₆₀; and (e, f) V₄₀-Catcher and M-V₄₀-Catcher. The size of M-V₄₀-Tag at 60° C. exceeded the limits of CONTIN algorithm (>10 μm). The vertical dashed lines at 20, 100, and 500 nm are added to aid the comparison of plots. All proteins were analyzed at 20 μM in PBS, except M-V₄₀-Tag (30 μM in PBS). Error bars are standard deviation of three measurements.

### DETAILED DESCRIPTION OF THE INVENTION

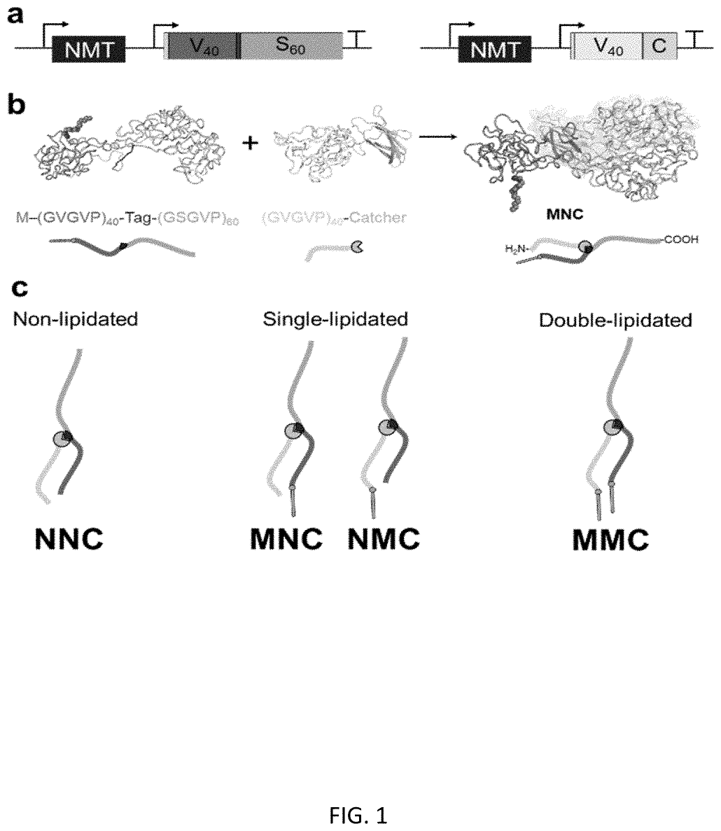

Referring to the figures, wherein like numerals refer to like parts throughout, there is seen in FIG. 1, the molecular design and synthesis of star amphiphiles according to the present invention. A topologically asymmetric building block was designed by selecting two different ELPs as hydrophobic and hydrophilic arms to control the extent of aggregation along the cylinder's main axis. Hydrophobic arms (A) contained 40 repeats of GVGVP (V₄₀)(SEQ ID NO: 1), while the hydrophilic arm (B) contained 60 repeats of GSGVP (S₆₀)(SEQ ID NO: 2). To create the branched topology, the SpyTag peptide was placed at the interface of the hydrophobic and the hydrophilic arms and the SpyCatcher was placed at the C-terminus of the second hydrophobic block (i.e., A-Tag-B and A-Catcher). Catcher/Tag pairs post-translationally form an isopeptide bond to create the core of the miktoarm star (A₂B). Both hydrophobic arms had free N-termini, while the hydrophilic arm contained a carboxylate group. The nonlipidated constructs are therefore referred to as NNC. Since myristoylation (M) occurs at the protein N-termini, three distinct SAFE constructs are biosynthetically accessible in this topology: one double-lipid (MMC) and two single-lipid (MNC and NMC) amphiphiles, which are distinguished by the location of the lipid. For MNC, “M” is attached to the hydrophobic arm linearly fused to the SpyTag sequence, while in NMC, it is attached to the hydrophobic arm linearly fused to SpyCatcher, as seen in FIG. 1. The hydrophobic arms can be modified with lipids such as myristic acid (shown and discussed above as an example) as well as cholesterol, farnesyl, geranylgeranyl, and phosphatidylethanolamine among others.

The present invention provides two main advantages. First, the present invention allows for the creation of lipidated proteins using a method that is much faster than traditional methods. Second, the present invention allows for the creation of thermally stable and mono-disperse nano worms. Although lipid modification of high molecular weight proteins has remained largely unexplored in materials science because most lipoproteins cannot be produced in E. coli as it lacks the enzymatic machinery, the present invention has overcome laborious, technically challenging, and low yield semi-synthetic approaches by genetically engineering E. coli to produce the desired protein and the minimum enzymatic machinery required for post-translational modification (PTM) with a specific lipid. Using this one-pot expression and modification system, the present invention can produce target lipoproteins at quantities sufficient for materials and biomedical applications. Using E. coli as a bio-factory according to the present invention also means that designer lipoproteins can be made from components not found in nature (such as the lipid described in this publication) because there is less interference from and to the metabolism of the host.

Recombinant Synthesis, Purification, and Molecular Characterization of SAFEs.

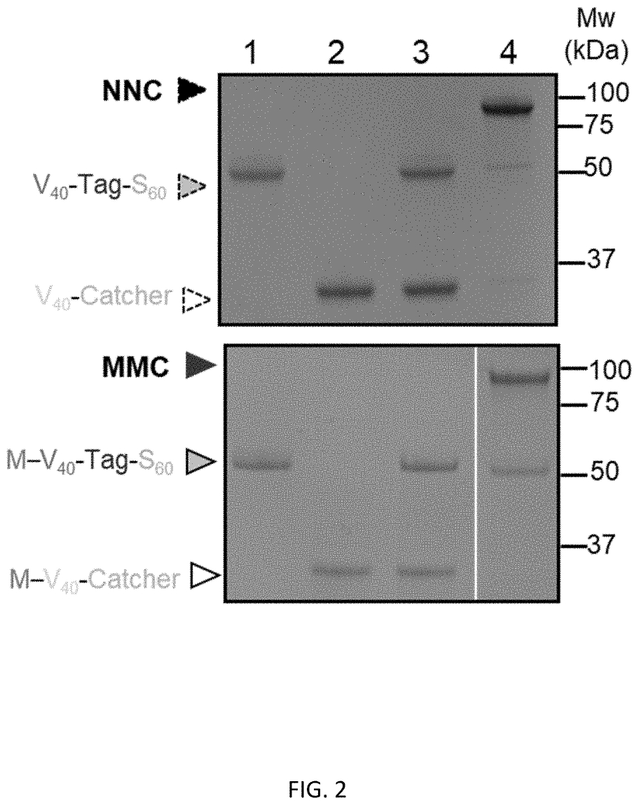

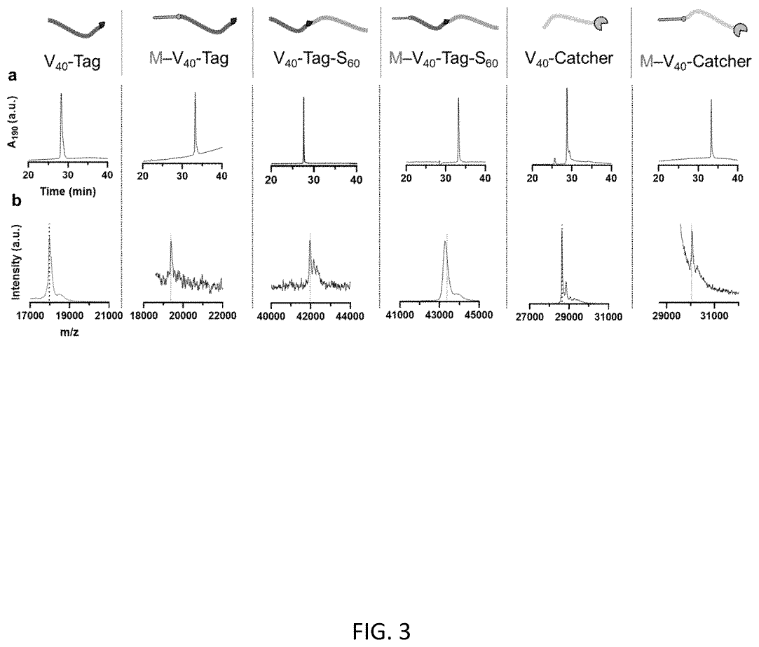

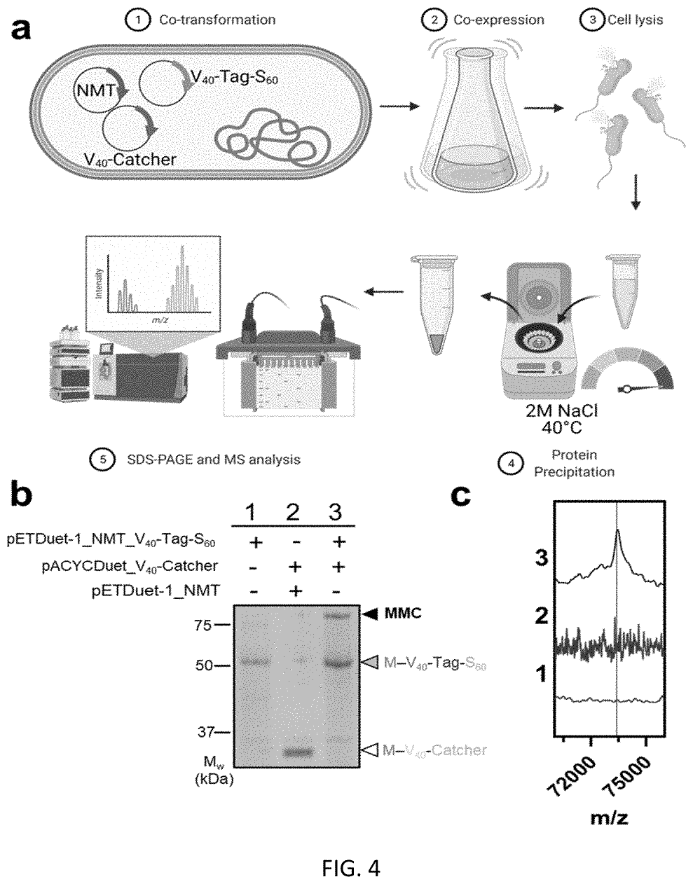

The genetic precision of SAFEs biosynthesis enables complete control over sequence and amphiphilic architecture with genetic precision. To do so, the necessary genetic elements were combined on two bicistronic plasmids (FIG. 1a, Table 1): 1) V₄₀-Tag-S₆₀; 2) V₄₀-Catcher; and 3) N-myristoyl-transferase enzyme (NMT) which lipidates the N-glycine of the hydrophobic arms when they are fused to a peptide substrate of NMT. These two plasmids can be used for recombinant expression and lipidation of individual components in separate cells. Combining these lipid-modified building blocks in the second step yields miktoarm stars with the desired lipidation pattern, as seen in FIG. 1B and FIG. 2). This two-pot method provided tight control over the production of constructs with asymmetric lipidated tails and was useful for generating the six linear controls, as seen in FIG. 3. To reduce the number of synthetic and processing steps, it is possible to biosynthesize constructs in one-pot by co-expression of NMT, V₄₀-Tag-S₆₀, and V₄₀-Catcher in one-cell, as seen in FIG. 4.

TABLE 1
Plasmids used to express different constructs.

| | Expressed from the | Essential features |
|---|---|---|
| Construct | vector^{[a]} | of the vector |
| V₄₀-Tag | pET24a(+)_VT | Kan^{r}, pBR322 Ori, |
| V₄₀-Tag-S₆₀ | pET24a(+)_VTS | monocistronic T7 |
| | | promotors |
| V₄₀-Catcher | pET24a(+)_VC | Kan^{r}, pBR322 Ori, |
| | | monocistronic T7 |
| | | promotors |
| | pACYCDuet-1_VC^{[b]} | Cm^{r}, p15A Ori, bicistronic |
| | | T7 promotors |
| M-V₄₀-Tag | pETDuet-1_NMT_rs- | Amp^{r}, pBR322 Ori, bicistronic |
| | VT | T7 promotors |
| M-V₄₀-Tag- | pETDuet-1_NMT_rs- | |
| S₆₀ | VTS | |
| M-V₄₀- | pETDuet-1_NMT_rs- | |
| Catcher | VC | |

^{[a]}rs: The peptide substrate for the NMT enzyme, which is the site of lipidation.
^{[b]}This plasmid is used for recombinant expression of MMC using a one-pot approach (FIG. 4).

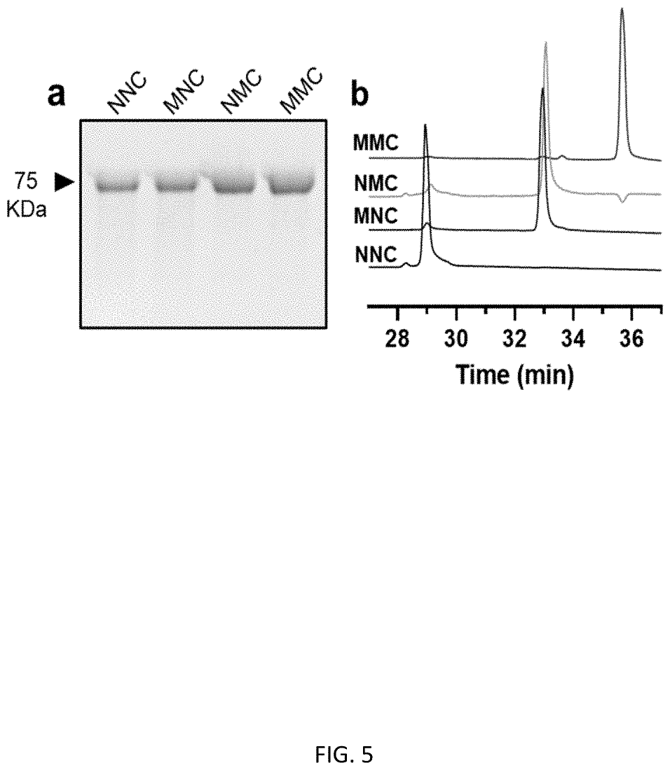

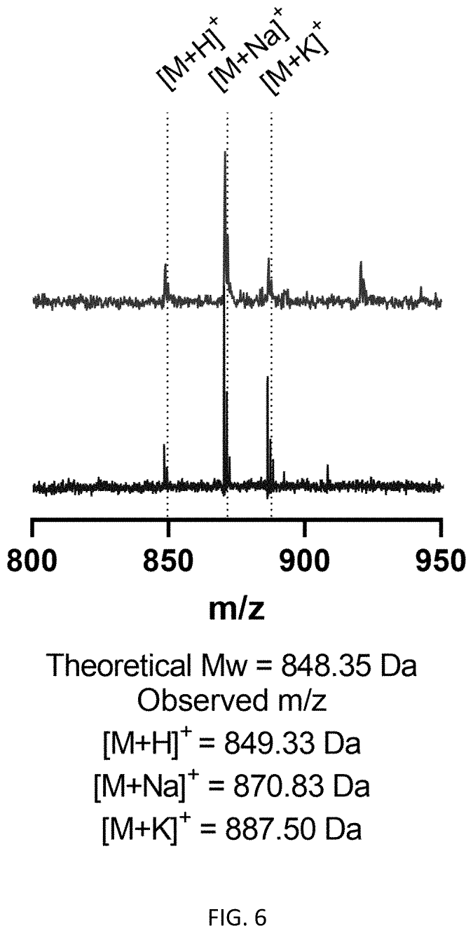

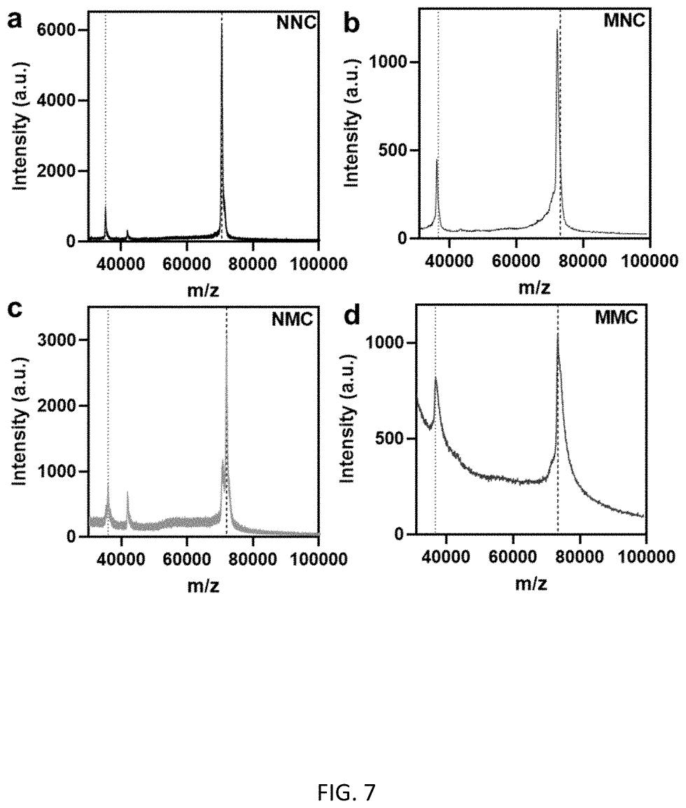

Each construct was purified by leveraging the temperature-triggered phase behavior of ELP arms and characterized using high-performance liquid chromatography and mass spectrometry to confirm purity and the regio- and chemoselectivity of modification, as seen in FIGS. 5, 6 and 7.

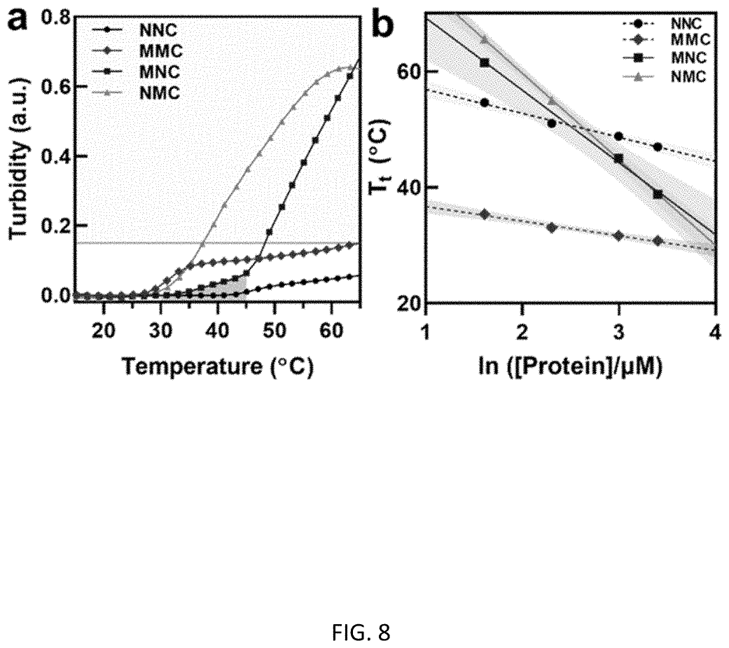

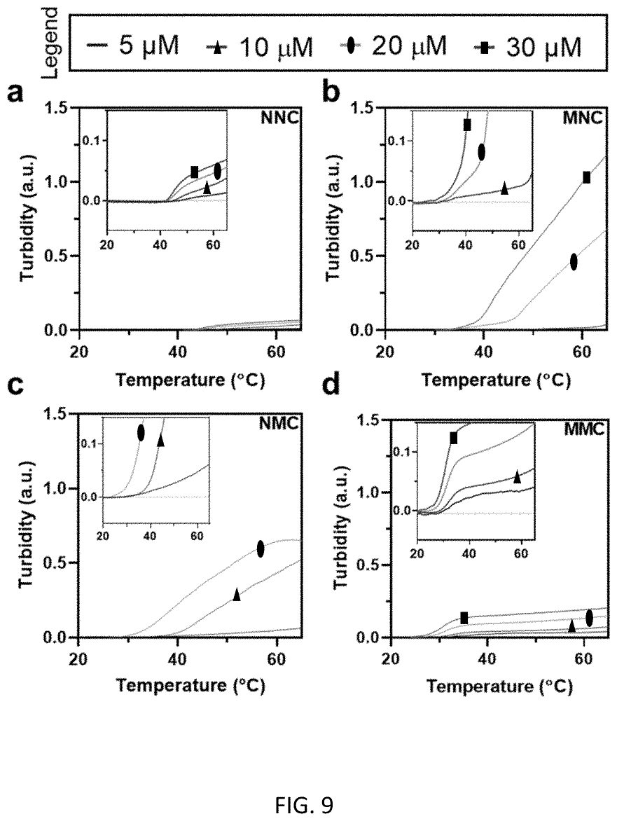

After purification, different biophysical and soft-matter characterization methods were used to test our hypothesis that the lipidation pattern modulates thermoresponse and nano-assembly of the SAFEs. Turbidimetry was first used to investigate the thermal response of SAFE constructs as a function of temperature and concentration, as seen in FIG. 8. The rationale was based on the observation that canonical ELPs exhibit a sharp lower critical solution temperature (LCST) phase transition. At T>LCST, the ELP-ELP interaction is more favorable than ELP-water, resulting in an attractive interaction that can drive the self-assembly of proteins. Thus, this operating parameter is critical for in vitro and in vivo applications as it links the assembly properties to the external temperatures.

Lipidation Pattern Modulates the Thermoresponse of SAFEs.

FIG. 8a depicts the turbidity profile of solutions of star amphiphiles (20 μM in phosphate buffer saline, PBS) as a function of temperature (15-65° C.). Lipidation pattern resulted in divergent turbidity profiles for SAFEs by changing 1) cloud point temperature (T_{cp}), the temperature at which turbidity starts to increase; 2) maximum solution turbidity (i.e., attenuation unit, AUₘₐₓ) at 65° C.; and 3) transition temperature (Tₜ), the inflection point as discussed in FIG. 8b. For instance, the T_{cp} was inversely correlated with the number of lipids attached to SAFEs: MMC˜25° C.<NMC and MNC˜30° C.<NNC˜45° C. Although the turbidity of NNC and MMC increased sigmoidally, both non- and double-lipidated constructs were noticeably transparent at elevated temperatures (AUₘₐₓ<0.15). In contrast, both single-lipid amphiphiles (MNC and NMC) were significantly more turbid (AUₘₐₓ>0.6), FIG. 8a, shaded area. Intriguingly, the behavior of single-lipid amphiphiles was also noticeably different from each other. Above T_{cp}, the turbidity of MNC increased linearly between 30-45° C. (shaded blue area) at which point the slope of turbidity vs. temperature increased 7-fold. In contrast, NMC showed a distinctly different behavior: Its turbidity profile exhibited a sigmoidal curve that was noticeably “shallower” than NNC, MMC, or canonical ELPs. These results suggest that the lipidation pattern modulates each construct's size and the kinetics of phase separation, as turbidity is caused by the scattering of incident light by SAFE assemblies. It may be inferred that single-tail constructs undergo liquid-liquid phase separation at elevated temperatures as the mesoscale coacervates strongly scatter visible light. On the other hand, the low turbidity of NNC and MMC is consistent with the formation of smaller nanoscale assemblies, as seen in linear ELP block copolymers above the LCST of the hydrophobic block.

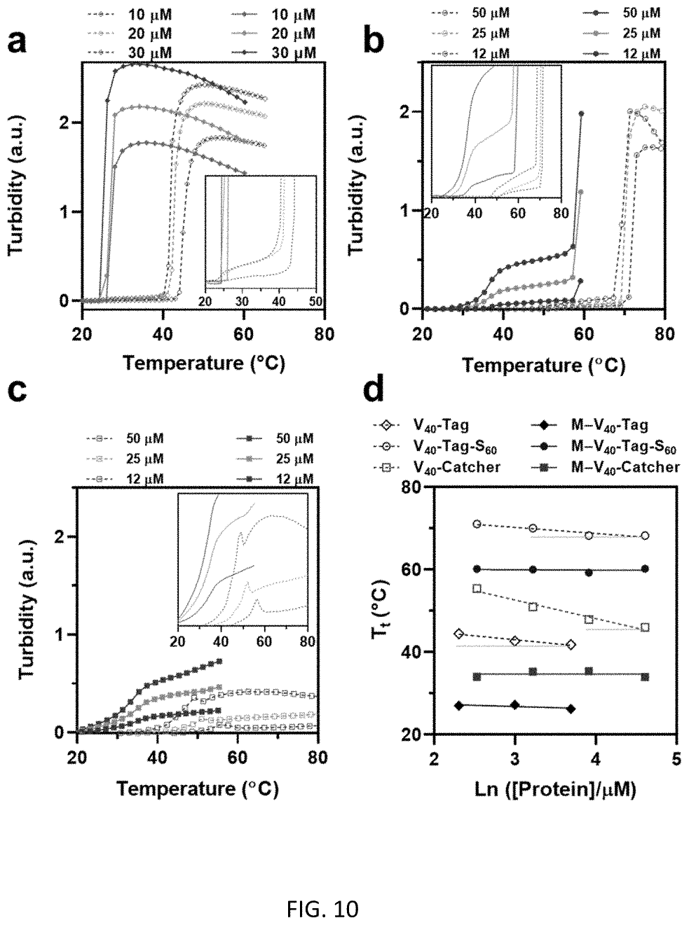

FIG. 8b shows that lipidation pattern also modulates the concentration dependence of Tₜ in SAFE constructs in the studied range (5-30 μM, FIG. 9). Notably, the Tₜ for MIVIC and NNC exhibits a lower concentration dependence than MNC and NMC (i.e., the slope of lines for NNC and MMC are −4.13 and −2.53° C. compared to −12.47 and −14.85° C. for MNC and NMC, respectively). Similarly, the Tₜ of linear nonlipidated (FIG. 10) controls exhibited a steep concentration dependence, while the LCST of the myristoylated controls was less dependent on concentration. This observation indicates the lipidation pattern modulate the inter-/intra-molecular nature of protein interactions that drive phase separation. Although quantitative models have been developed to predict the LCST of linear ELPs and their block copolymers as a function of molecular features and solution conditions (i.e., the polarity of the guest residue, ELP length, concentration, and ionic strength, etc.), the influence of nonproteinogenic motifs (lipid) or nonlinear topologies (branched, dendritic, etc.) is less well understood. Additional work is needed to elucidate these principles in non-canonical systems.

Results of turbidity experiments revealed two insights: 1) The thermal behavior of the symmetric constructs NNC and MMC were noticeably different from the asymmetrically lipidated constructs MNC and NMC. 2) Constructs that were identical except for lipid location (i.e., MNC vs. NMC) have divergent thermo-responses. It is hypothesized that these observed differences originate from the temperature-dependent assembly of SAFEs into different nano-/mesoscale structures.

Lipidation Patterns Modulate the Nano- and Mesoscale Assembly of SAFEs.

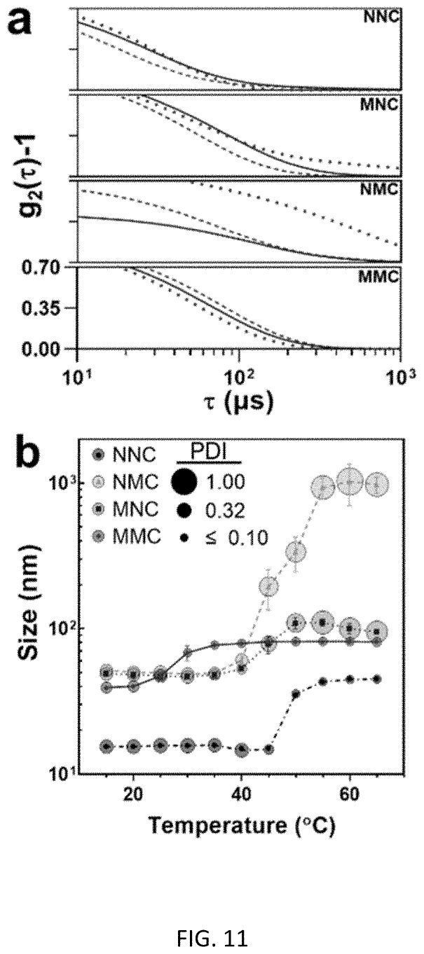

To test this hypothesis, dynamic light scattering (DLS) was performed to probe the assembly of SAFE in PBS as a function of temperature (15-65° C. at 5° C. increments). FIG. 11 shows the autocorrelation functions (ACF) recorded at 20, 40, and 60° C. for each construct (blue, purple, and red, respectively). The comparison of two key features of ACFs, decay time and mode (i.e., the intersection with x-axis and mono-/bi-phasic decay), confirms that lipidation pattern alters the assembly and temperature-responsiveness of SAFE assemblies without making assumptions about the shape of the aggregates.

At 20° C., the mono-modal decay time of NNC (t<10² ms) was consistent with the presence of unimeric or unassembled species, while all lipidated constructs formed nanoscale assemblies (10² ms<t<10³ ms). Consistent with turbidimetry results, the ACFs for NNC and MMC shifted only slightly at higher temperatures (40 and 60° C.), but both constructs remained in the nanoscale range. The behavior of single-lipid constructs was noticeably different, as nanoscale assemblies at 20° C. formed much larger aggregates at 60° C., t>10³ ms consistent with the size regime in the micron range. In addition, we observed subtle differences between the temperature-dependent nano-assembly of NMC and MNC. The NMC assemblies remained in the nanoscale range at 20 and 40° C. and formed larger micron-sized aggregates at 60° C. In contrast, the ACF of MNC deviated from one-phase exponential decay, which indicates the formation of a mixture of small and large particles.

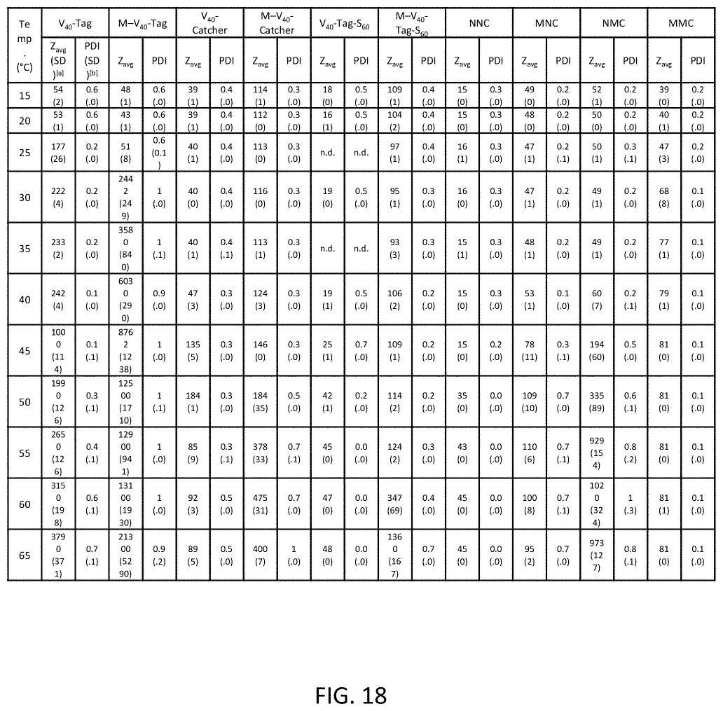

Next, the ACFs were analyzed using the cumulant method to derive the size and dispersity (Z_{avg} and polydispersity index, PDI) of SAFE assemblies at various temperatures. FIG. 11 (and the table in FIG. 18) shows the results of this analysis as a bubble plot with the center of each circle representing Z_{avg}, and the area of each circle representing PDI. Z_{avg} is the intensity-weighted mean hydrodynamic size of the ensemble collection of particles, and PDI represents the dispersity of this ensemble (0 (monodisperse)<PDI<1 (polydisperse)).

As shown in FIG. 11b, the size of unmodified stars (Z_{avg}=12 nm, black lines and circles) at low temperatures (<45° C.) suggests a lack of assembly in this range. Above 50° C., Z_{avg} increased to ˜30 nm, indicating the formation of small assemblies at higher temperatures. The low PDI of these samples suggests that they are spherical, consistent with the formation of micellar assemblies observed in canonical ELP-based block copolymers. The single-lipid NMC and MNC formed assemblies of similar sizes and PDI at low temperatures (blue and green lines and bubbles). As T>T_{cp}, both samples started to form larger aggregates, but their behavior started to diverge. The Z_{avg} for NMC exceeded 1 μm, while the Z_{avg} of MNC was significantly smaller (˜100 nm). This is consistent with the formation of coacervates for NMC, though it reflects the unequal contribution of small and large MNC particles. Meanwhile, the behavior of MMC was distinctly different.

At low temperatures, MMC assembled into aggregates with an average size of 30 nm and a lower PDI compared to NMC and MNC. As T>T_{cp}, the aggregate size started to increase and reached ˜80 nm at 30° C. Increasing the temperature to 65° C. did not result in a significant increase in aggregate size. Notably, the PDI of single-lipid amphiphiles increased with temperature (approaching the maximum theoretical value of 1), while the PDI of MMC decreased with temperature. These results were interpreted as indicating the formation of a more homogenous assembly population for MIVIC, which drastically contrasts with the observed increase in the polydispersity of NMC and MNC at higher temperatures.

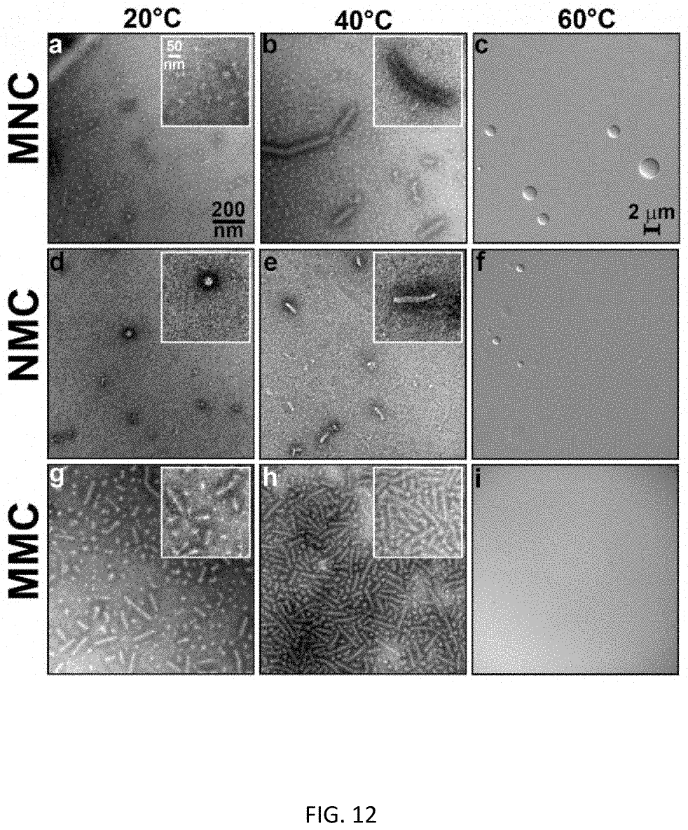

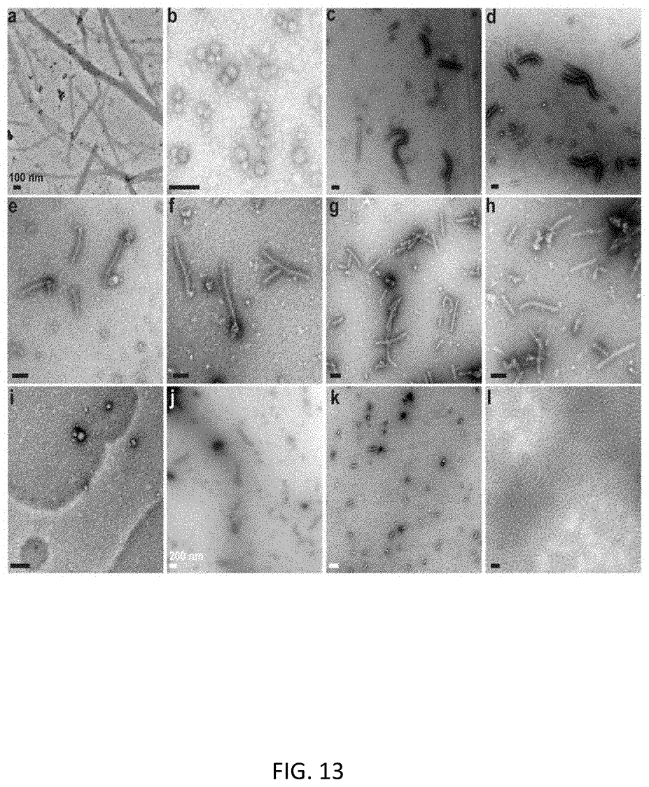

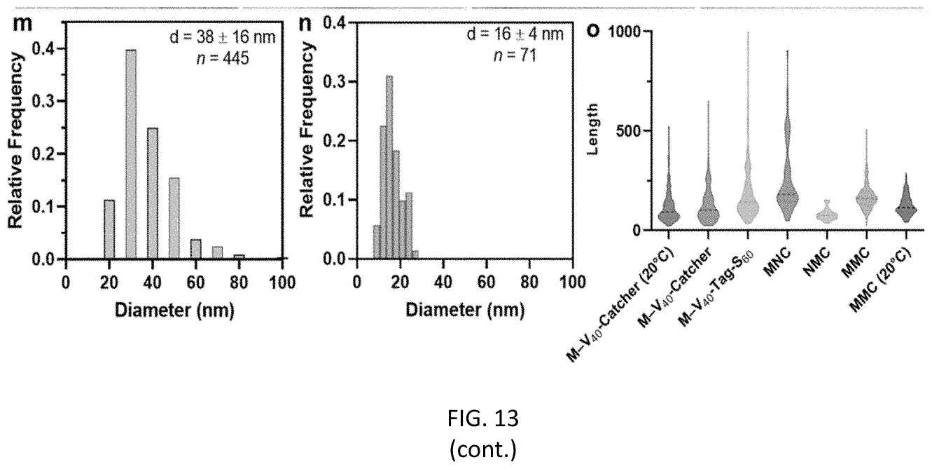

To complement insights provided by turbidity and DLS, microscopy was performed to visualize the assembly of different constructs at different temperatures (FIG. 12). Transmission electron microscopy (TEM) was used to characterize the nanoscale assemblies. Consistent with DLS, NNC only formed small spherical assemblies at elevated temperatures (16±4 nm, FIG. 13). All lipidated constructs formed temperature-responsive nano-assemblies. MNC formed a mixture of isotropic spherical aggregates and ill-defined high-aspect-ratio structures at 20° C. (FIG. 12a). Increasing the temperature to 40° C. resulted in supramolecular bottle-brush assemblies with a narrow core (white area) and a dense brush layer (darker area), as shown in FIG. 12b. In contrast, NMC predominantly formed short worm-like micelles at 20° C. which transitioned into nano-tape structures at 40° C. (FIG. 12e). The cores in these tapes were noticeably larger than those of bottle brushes, while their corona was less visible when compared to the brush-like structures. Meanwhile, MMC first assembled into a mixture of spherical particles and nanoworms at 20° C. As the temperature increased to 40° C., the number and length of nanoworms increased at the expense of spherical aggregates, consistent with the reduction of PDI observed in DLS, as seen in FIG. 13.

Differential interference contrast (DIC) microscopy confirmed the effect of lipidation patterns on the mesoscale assembly of SAFE constructs. Both single-lipid amphiphiles underwent liquid-liquid phase separation and formed micron-size coacervates at 60° C. (FIGS. 12c and f). In contrast, MMC did not undergo bulk-phase separation from the solution, and no coacervates were observed, as seen in FIG. 12i.

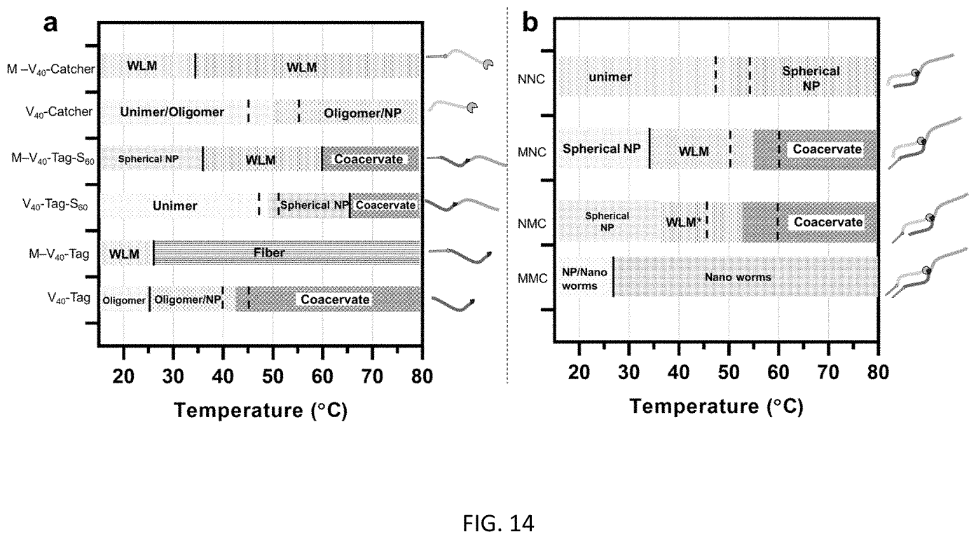

Results of turbidimetry, scattering, and microscopy experiments consistently demonstrate the following points: 1) Lipidation pattern changes the assembly and thermoresponse of SAFEs. 2) The changes in material properties as a function of temperature for the non- and double-lipidated constructs (NNC and MMC) differ considerably from the behavior of single-lipid SAFEs (MNC and NMC). 3) Intriguingly, differences in the lipidation site resulted in subtle differences in the assembly and thermoresponse of single-lipid amphiphiles, as seen in FIG. 14.

These findings confirm the hypothesis that the material properties of SAFE can be modulated by changing their lipidation patterns and amphiphilic architecture. However, they also hint at a complex interplay between lipidation pattern, structure, and energetics of chemically and topologically modified SAFEs. These observations motivated our use of MD simulations to gain molecular-level insight into the interplay between the physicochemistry of lipids and the composition of the various constructs. To compute in silico properties, we focused on unimer dynamics that are precise yet have relatively low computational cost, while being mindful that thermoresponse and assembly are bulk properties (i.e., impacted by interactions between multiple chains). However, past studies have shown that single-chain properties such as hydration can reliably predict LCST behavior for linear ELPs. Similarly, we suggest that the physicochemical interplay between and among protein, lipid, and branching point modulate the key drivers of bulk properties at the single-chain level. The MD simulations were used to compute a series of structural and physicochemical properties corresponding to the size, shape, and hydration of constructs at 5, 37, and 67° C.

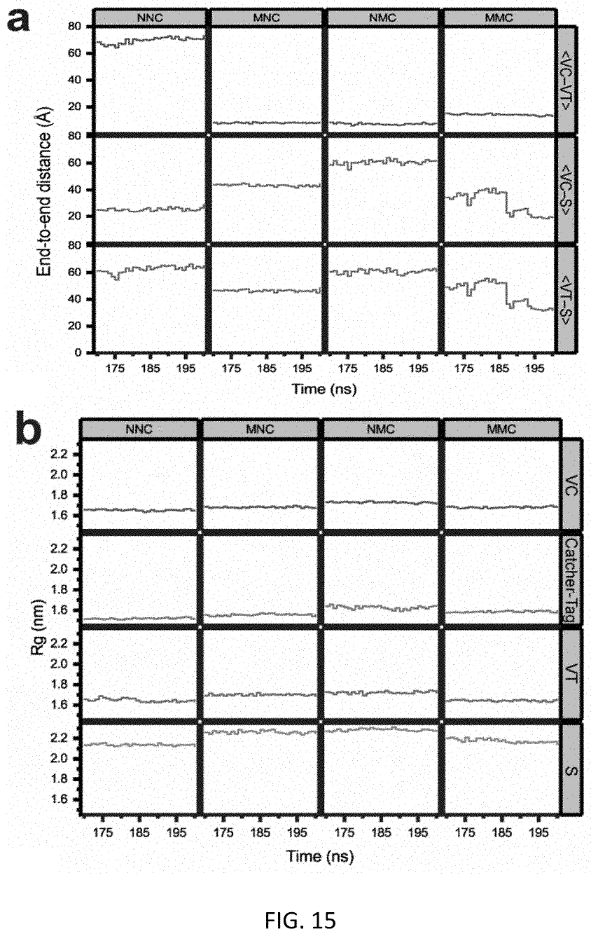

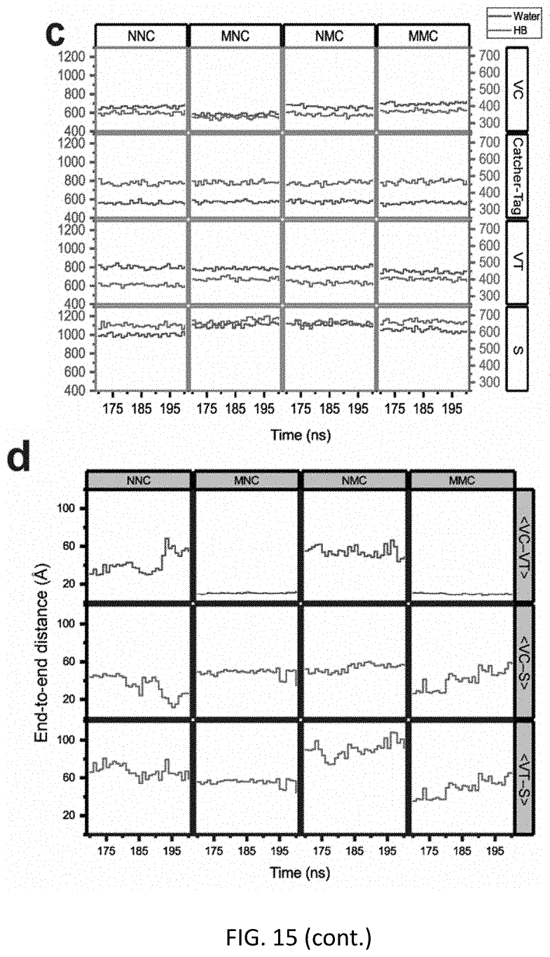

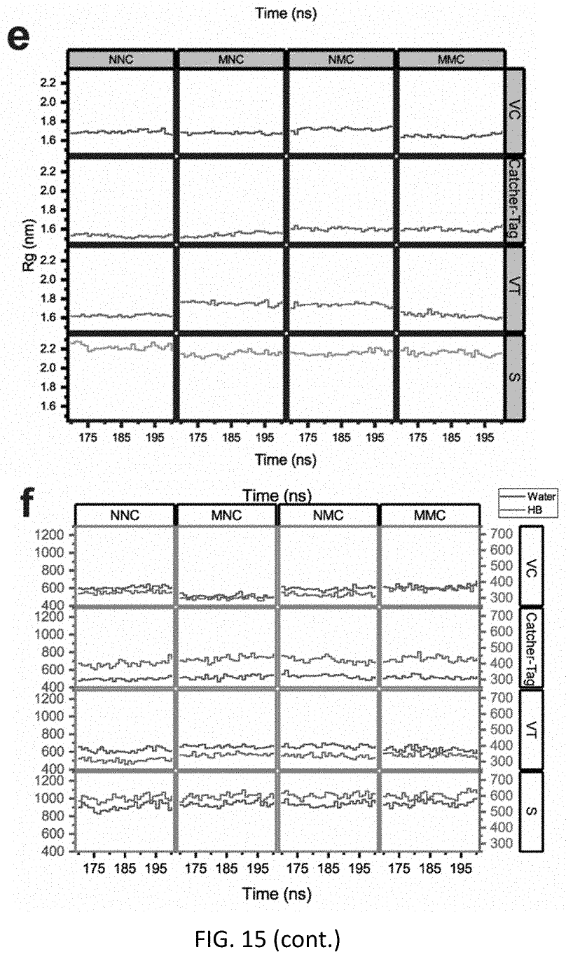

The trajectories obtained in the last 200 ns of MD simulations were used to derive 15 parameters related to different aspects of amphiphilic architecture (size, form(shape), and hydration) from the trajectories at 100 ps intervals (FIG. 15). These parameters include: a) radius of gyration (R_{g}) of each arm and the branching point; b) end-to-end distance between the three arms; c) the number of water molecules in the first hydration shell of the molecule (3.2 Å cutoff); and d) hydrogen bonds between the protein and water, as seen in Table 2 below:

TABLE 2
Definitions and categorization of molecular features extracted from MD
simulations as PCA input.

| | Parameter | Unit | Definition | Category |
|---|---|---|---|---|
| 1 | <VC-VT> | Å | Root-mean-square end-to-end distance | Form |
| | | | between VC and VT blocks | |
| 2 | <VC-S> | Å | Root-mean-square end-to-end distance | Form |
| | | | between VC and S blocks | |
| 3 | <VT-S> | Å | Root-mean-square end-to-end distance | Form |
| | | | between VT and S blocks | |
| 4 | R_{g} (VC) | nm | Radius of gyration of VC block | Size |
| 5 | R_{g} (C) | nm | Radius of gyration of branching point | Size |
| | | | (SpyCatcher-Tag complex) | |
| 6 | R_{g} (VT) | nm | Radius of gyration of VT block | Size |
| 7 | R_{g} (S) | nm | Radius of gyration of S block | Size |
| 8 | V_{C} <W> | N/A | average number of water molecules | hydration |
| | | | in the first hydration layer of V_{C} | |
| 9 | V_{C} <HB> | N/A | average number of hydrogen bonds | hydration |
| | | | (HB) between the V_{C} and water | |
| 10 | C <W> | N/A | average number of water molecules | hydration |
| | | | in the first hydration layer | |
| | | | of branching point | |
| 11 | C <HB> | N/A | average number of HB between the | hydration |
| | | | branching point and water | |
| 12 | VT <W> | N/A | average number of water molecules | hydration |
| | | | in the first hydration layer of V_{T} | |
| 13 | VT <HB> | N/A | average number of HB between the | hydration |
| | | | VT and water | |
| 14 | S <W> | N/A | average number of water molecules | hydration |
| | | | in the first hydration layer of S | |
| 15 | S <HB> | N/A | average number of HB between S | hydration |
| | | | and water | |

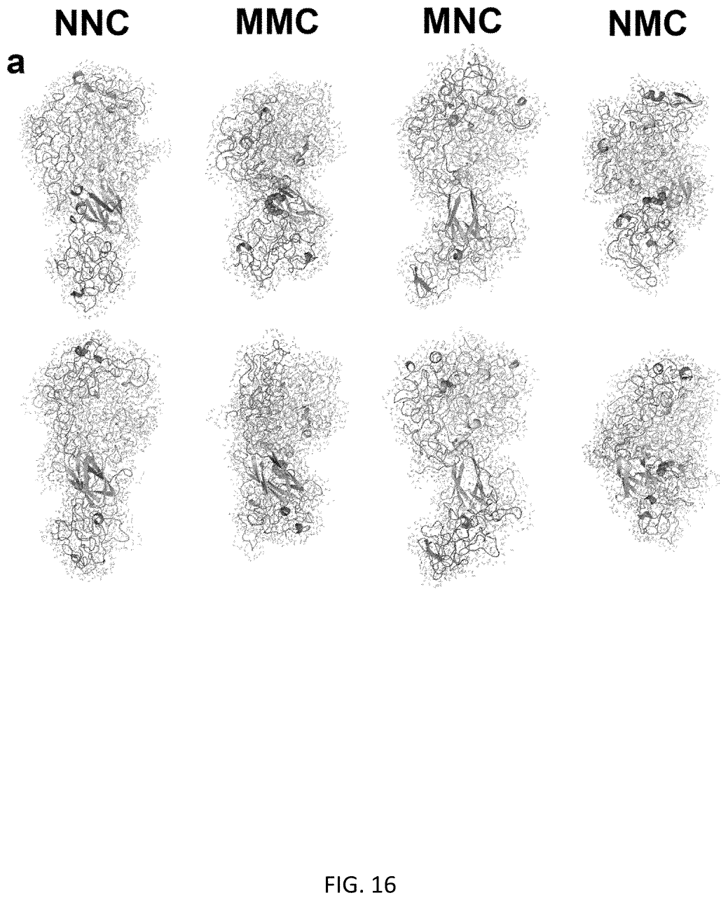

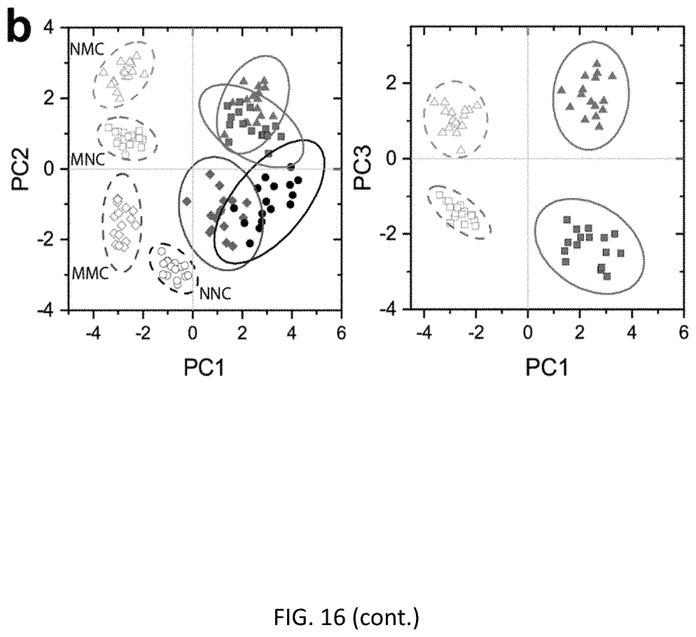

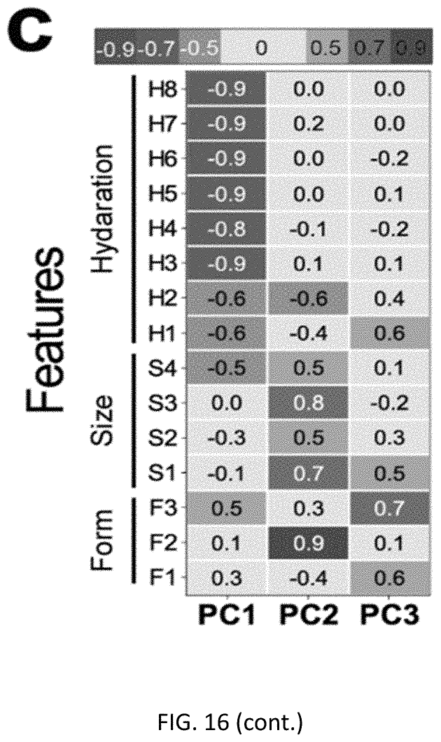

The equilibrium structures of NNC, MNC, NMC, and MMC show how the single-tail and double-tail modifications alter the intramolecular structure of the constructs, as seen in FIG. 16. Principal component analysis (PCA), an unsupervised machine learning (ML) algorithm, was then used for clustering the simulation output parameters in a space defined by the first three principal components (PCs), which accounted for at least 75% of the variation in the original dataset. As shown in FIG. 16, constructs with different amphiphilic architectures were separated into nonoverlapping areas of space defined by these PCs. Specifically, PC1 was strongly correlated with the effect of temperature, as the clusters for all constructs shift to the right as the temperature is increased. Moreover, PCI could discriminate between nonlipidated and lapidated constructs. PC2 captured differences between single-lipid constructs and non- or double-lipidated constructs, MNC/NMC vs. NNC/MMC. PC3 discriminated the lipidated constructs as well as the single-lipid constructs (MNC vs. NMC). The separation between these clusters, which is consistent with experimental findings, strongly supports the notion that single-chain simulations can capture the effect of lipids and temperature on the structure, hydration, and energetics of SAFE constructs. Moreover, these results demonstrate that the combination of MD simulations ML algorithms can detect subtle differences in the behavior of highly homologous amphiphiles, which should facilitate the design of soft materials.

Because PCs include the varied influences of the original features, it is possible to trace differences between the clusters to back to changes in these features as a function of amphiphilic architecture or temperature. This information is captured in loading plots (FIG. 16), which elucidates the contribution of features to each PC on a normalized scale, −1 to 1. For instance, features corresponding to hydration are negatively correlated with PC1. As temperature is increased (along the PC1 positive axis), constructs are dehydrated. This correlation is intuitive given the LCST behavior of ELP and is consistent with previous computational studies. A detailed analysis of loading plots also revealed the subtle biophysical interplay between the different components of the molecular syntax. For example, dehydration of the hydrophobic arm fused to SpyCatcher showed a weaker correlation with temperature (cf. loading of H1-2 with H3-8 in PC1, −0.6 vs. −0.9), but dehydration uniquely contributed to PC2 and 3, which discriminated between SAFEs with different lipidation pattern.

Extending this analysis to other features enables us to parse the contribution of lipidation patterns to the observed differences between the constructs and identify similar intuitive and subtle variations in size, shape, or hydration of each construct with high resolution. An analogy to the “packing parameter,” which predicts the assembly of amphiphiles based on geometric considerations such as size and shape of hydrophobic/hydrophilic moieties, is illustrative. The lipidation pattern influences the physicochemical characteristics of arms—size, hydrophobicity, and shape of various domains at the unimer level even when they are distant from the lipidation site. These variations can explain the observed differences in the nanoscale assembly of these amphiphiles as the function of molecular syntax or temperature.

Discussion

Controlling the length of 1D cylindrical assemblies—a prerequisite for formation of stable NWs—requires balancing the delicate interactions of building blocks along the main axis versus the endcap region. For most amphiphiles, the addition of a monomer to the cylinder length is a noncooperative process. That is, the free energy of micelle growth does not change as the aggregation number is increased. This property hinders the thermodynamic control over the growth process and promotes the formation of a polydisperse mixture with lengths ranging from nano-to-micrometer. These difficulties may explain why there are few systematic investigations on the preparation of protein NWs in the literature.

It was hypothesized that changing the linear topology of protein fusions may broadens the stability of nanoworms in the phase diagram. To test this hypothesis, two types of PTMs, lipidation and branching, were combined to synthesize high molecular weight, sequence-defined star amphiphiles with unique, and programmable amphiphilic architecture defined by the composition of proteins and the lipidation patterns. It was demonstrated that lipidation pattern modulates the phase behavior of star amphiphiles. Intriguingly, the addition of a single lipid reduced the LCST phase boundaries and promoted macroscopic phase separation <40° C. In contrast and counter-intuitively, double-lipidated constructs did not show macroscopic phase separation even when heated to 65° C. While LCST phase behavior is a useful feature for scalable purification of proteins, it also presents an upper operating condition for using these constructs as nanomaterials, since above the cloud point, mesoscale coacervates are formed, as seen in FIG. 14. This limits the use of lipid-modified elastin for high-temperature applications such as templated synthesis of nanomaterials. Therefore, the results for MMC may provide a translatable design principle for maintaining the solubility of lipidated constructs even at very high temperatures by avoiding the LCST transition into micron-sized aggregates.

Similarly, DLS and TEM confirmed that lipidation pattern significantly influences the nanoscale assembly of star amphiphiles as a function of temperature. Importantly, the present invention shows that a judicious choice of amphiphilic architecture can be used to prepare adaptive nanoworms that undergo a shape transformation in response to temperature stimuli. This morphological change, combined with the modulation of phase separation behavior discussed above, increases the NW stability even at extremely high temperatures. These characteristics are not found in the protein NW literature; nano-assembly either did not change with temperature or formed large aggregates at elevated temperatures.

To understand the origin of these divergent behaviors, MD simulations of a unimer were combined with PCA to parse the effect of lipidation patterns on the energetics and structure of these hybrid amphiphiles. MD simulations are increasingly being utilized to provide molecular-level insights that are experimentally unattainable and to explain dynamical behavior observed in self-assembled nanostructures. The power of MD simulations lies in their ability to account for the effects of complex sequence-encoded interactions.

Conclusions

This present invention provides several notable outcomes: First, it provides a straight-forward roadmap to synthesize adaptive, recombinant NW. Due to their amphiphilicity, these NWs can easily solubilize hydrophobic chemotherapeutics without resorting to complex, inefficient, and time-consuming conjugation/purification protocols. The recombinant nature of this system enables the fusion of genetically encoded bioactive or targeting peptides, which can be used to optimize the delivery and efficacy of these nanoplatforms.

Second, is the significant expansion of the hybrid protein-based-material design space by demonstrating the compatibility between two classes of PTMs, lipidation and protein branching. These methods should be generalizable to other classes of proteins and PTMs. Thus, this work will advance the study and design other hybrid systems, such as lipidated resilin with upper critical solubility phase behavior, or proteins modified with other classes of lipids (e.g., cholesterol) or charged PTMs such as phosphorylation.

Third, integration of experiment, simulation, and data analytics provides a road map to move synthesis of hybrid functional biomaterials beyond current ad hoc approaches into the realm of predictive design. Traditional brute-force material design, synthesis, and characterization strategies to elucidate the design principles of these hybrid materials are impractical given the large design space resulting from the orthogonality of protein, lipidation, and branching “building blocks.” The proposed alternative strategy is to use MD simulations and data analytics to survey quickly and less expensively the hybrid design space and then experimentally verify results. While commonly used in biophysical and biochemical studies, MD simulations is an emergent tool to design soft materials. However, realizing the full potential of this method, requires new approaches to reduce the computational cost of multiscale modeling required to predict the properties of desired materials. As shown here, the integration of machine learning can provide insights into design principles—a thermodynamically grounded understanding of the contribution of molecular syntax to programmable assembly of hybrid materials. Elucidating these principles will foster the development of next-generation biomaterials and therapeutics whose forms and functions rival the exquisite hierarchy and capabilities of biological systems.

### EXAMPLE

### 1. Materials

Restriction enzymes, ligase, corresponding buffers, DNA extraction and purification kits, and chemically competent Eb5alpha and BL21(DE3) cells were purchased from New England Biolabs (Ipswich, Mass.). DNA oligonucleotides and gene fragments were synthesized by Integrated DNA Technologies (Coralville, Iowa). Apomyoglobin, adrenocorticotropic hormone (ACTH), sinapinic acid, alpha-cyano-4-hydroxycinnamic acid, ammonium bicarbonate, and trifluoroacetic acid (TFA) were purchased from Sigma-Aldrich (St. Louis, Mo.). High-performance liquid chromatography-(HPLC) grade acetonitrile, isopropyl β-D-1-thiogalactopyranoside (IPTG), SnakeSkin™ dialysis tubing with 7 K nominal molecular weight cut off (MWCO), mass spectroscopy grade Pierce™ trypsin protease, tryptone, yeast extract, agar, sodium chloride, ampicillin, phosphate buffer saline (PBS), myristic acid, urea, and ethanol were purchased from Thermo Fisher Scientific (Rockford, Ill.). Mini-PROTEAN® TGX Stain-Free™ Precast Gels, Precision Plus Protein™ All Blue Pre-stained Protein Standard, and Precision Plus Protein™ Unstained Protein Standards were purchased from Bio-Rad Laboratories, Inc. (Hercules, Calif.). The carbon-coated grid (CF300-Cu) was purchased from Electron Microscopy Sciences (Hatfield, Pa.). Deionized water was obtained from a Milli-Q® system (Millipore SAS, France). Simply Blue™ SafeStain was purchased from Novex (Carlsbad, Calif.). All chemicals were used as received without further purification.

### 2. Cloning, DNA, and Proteins' Sequences

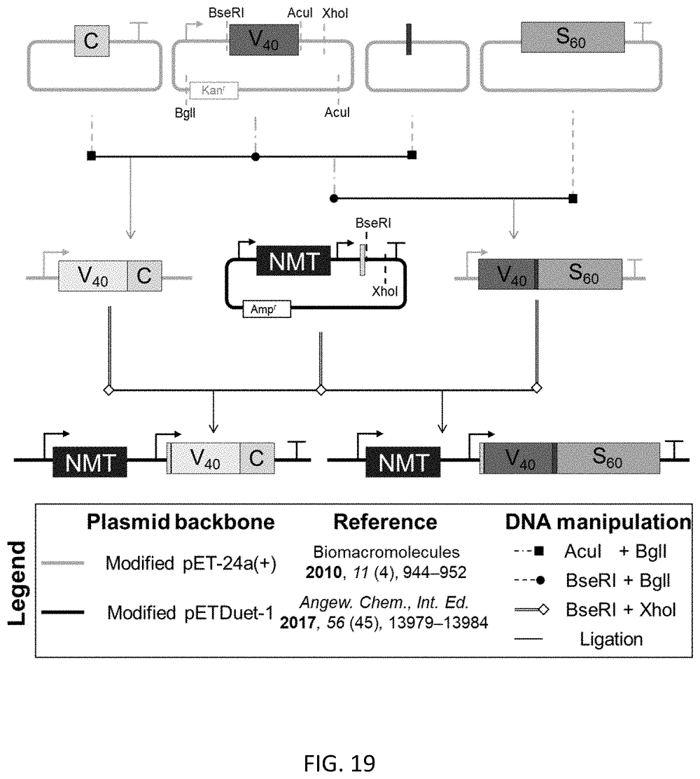

The genes encoding SpyTag and SpyCatcher sequences were first cloned into modified pET24a(+) using restriction digest and NEBuilder® HiFi DNA Assembly. The modified pET24a(+) vector contains unique recognition sequences for type IIs restriction enzymes BseRI and Acul that flank the gene of interest. This feature enables the directional and modular assembly of repetitive protein polymers, i.e., (GVGVP)₄₀ (SEQ ID NO: 1) and (GSGVP)₆₀ (SEQ ID NO: 2) and the Spy pairs. In parallel, we first fused the (GVGVP)₄₀ (SEQ ID NO: 1) gene to the N-termini of SpyTag and SpyCatcher genes. The (GVGVP)₄₀-Tag (SEQ ID NO: 1) was subsequently fused to the N-terminus of (GSGVP)₆₀ (SEQ ID NO: 2) in the second round of directional ligation to generate plasmids encoding for linear blocks. To generate myristoylated constructs, these genes were than subcloned into a modified pETDuet-1, a bicistronic vector containing all the necessary genetic elements for N-myristoylation. These elements include the NMT enzyme from S. cerevisiae and the site of N-myristoylation: a peptide substrate derived from ARF2 protein. The linear blocks were subcloned downstream of ARF2 recognition sequence (underlined). The schematic of this process is shown in FIG. 19. Control plasmids lacking NMT and RS were used to express nonmyristoylated proteins, as see in Table 1 above.

```
V₄₀-Tag-S₆₀
(SEQ ID NO: 3)
GLYASKLFSNLGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGAHIVMVDAYKPTKGSGVPGSGVPGSGVPGSGVPGSGVP
GSGVPGSGVPGSGVPGSGVPGSGVPGSGVPGSGVPGSGVPGSGVPGSGVP
GSGVPGSGVPGSGVPGSGVPGSGVPGSGVPGSGVPGSGVPGSGVPGSGVP
GSGVPGSGVPGSGVPGSGVPGSGVPGSGVPGSGVPGSGVPGSGVPGSGVP
GSGVPGSGVPGSGVPGSGVPGSGVPGSGVPGSGVPGSGVPGSGVPGSGVP
GSGVPGSGVPGSGVPGSGVPGSGVPGSGVPGSGVPGSGVPGSGVPGSGVP
GSGVPGSGVPGSGVPGSGVPGSGVPGY
V₄₀-Catcher
(SEQ ID NO: 4)
GLYASKLFSNLGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVGV
PGVGVPGVGVPGVDTLSGLSSEQGQSGDMTIEEDSATHIKFSKRDEDGKE
LAGATMELRDSSGKTISTWISDGQVKDFYLYPGKYTFVETAAPDGYEVAT
AITFTVNEQGQVTVNGKATKGDAHIG
SpyTag
(SEQ ID NO: 5)
Forward 5′- C GCA CAC ATA GTA ATG GTA GAC GCC
                        TAC AAG CCG ACG AAG GGC TAA TGA TAA
                        TGA TCT TCA G -3′
Reverse 3′- CCG CGT GTG TAT CAT TAC CAT CTG CGG
                          G A H I V M V D A
                        ATG TTC GGC TGC TTC CCG ATT ACT ATT
                          Y K P T K G * * *
                        ACT AGA AGT CCT AG -5′
                          * S S G
SpyCatcher
(SEQ ID NO: 6)
ATGGGCGTTGATACCTTATCAGGTTTATCAAGTGAGCAAGGTCAGTCCGG
TGATATGACAATTGAAGAAGATAGTGCTACCCATATTAAATTCTCAAAAC
GTGATGAGGACGGCAAAGAGTTAGCTGGTGCAACTATGGAGTTGCGTGAT
TCATCTGGTAAAACTATTAGTACATGGATTTCAGATGGACAAGTGAAAGA
TTTCTACCTGTATCCAGGAAAATATACATTTGTCGAAACCGCAGCACCAG
ACGGTTATGAGGTAGCAACTGCTATTACCTTTACAGTTAATGAGCAAGGT
CAGGTTACTGTAAATGGCAAAGCAACTAAAGGTGACGCTCATATTGGCTA
ATGATAATGA
```

### 3. Protein Expression and Purification

All proteins were expressed in E. coli BL21(DE3) strains with IPTG-inducible lac promotors. The expression and purification of myristoylated proteins followed a previously established protocol modified by reducing induction time to 6 h. Cells were harvested by centrifugation (3745×g, 30 min, 4° C.), resuspended in PBS, 10 mL per 1 L of culture), and lysed by sonication. The lysate was clarified by centrifugation (22,830×g, 4° C., 15 min) before purification of proteins using inverse transition cycling (ITC). Proteins used for self-assembly studies were purified by reversed-phase HPLC (RP-HPLC) to ensure >95% purity. Organic solvents were removed by dialyzing the protein solution against water using SnakeSkin™ Dialysis Tubing (MWCO 7 kD) for ˜18 hours. The proteins were then lyophilized and stored at −20° C.

Methods

Cloning. Genes encoding linear building blocks V₄₀-Tag-S₆₀ and V₄₀-Catcher were constructed using Gibson assembly and recursive directional ligation by plasmid reconstruction. The identity of each gene was confirmed using Sanger sequencing.

Protein Expression and Purification. Proteins were expressed in E. coli BL21(DE3) grown in 2× YT medium under the control of lac promotor. Myristoylated proteins were expressed in 2× YT the medium, supplemented with myristic acid (100 mM). All proteins were first purified by exploiting the lower critical solubility behavior of ELP followed by reversed phase HPLC to ensure >95% purity before self-assembly studies.

Synthesis of star amphiphiles. Miktoarm star amphiphiles were synthesized by mixing the corresponding linear building blocks (ELP block copolymer and ELP-Catcher fusions) in reaction buffer (PBS or PBS supplemented with 4M urea), and incubation at room temperature for 2 h. For instance, MMC was synthesized by reacting M-V₄₀-Tag-S₆₀ (30 μM) with M-V₄₀-Catcher (20 μM). Reaction progress was monitored using SDS-PAGE and the appearance of the product band (˜75-100 kDa) and reduction in the intensity of starting material bands (˜50 kDa and ˜37 kDa), as seen in FIG. 2. Star amphiphiles were subsequently purified to homogeneity using RP-HPLC.

RP-HPLC. Analytical and preparative RP-HPLC were performed on a Shimadzu instrument equipped with a photodiode array detector on C18 columns (Phenomenex Jupiter® 5 μm C18 300 Å, 250×4.6 mm and 250×10 mm). The mobile phase was a linear gradient of acetonitrile and water (0 to 90% acetonitrile over 40 min, each phase supplemented with 0.1% TFA).

MALDI-TOF-MS. Matrix-assisted laser desorption/ionization, time-of-flight mass mass spectrometry (MALDI-TOF-MS) was conducted on a Bruker Autoflex III. N-terminal peptide fragments were characterized after digestion with trypsin.

Turbidimetry Assay. The thermal behavior of proteins was characterized using a Cary 100 UV-Vis Spectro-photometer (Agilent, Santa Clara, Calif.) equipped with a Peltier temperature controller. The optical density of the solution at 350 nm was recorded at 15-65° C. while heating the solution at the rate of 1° C./min.

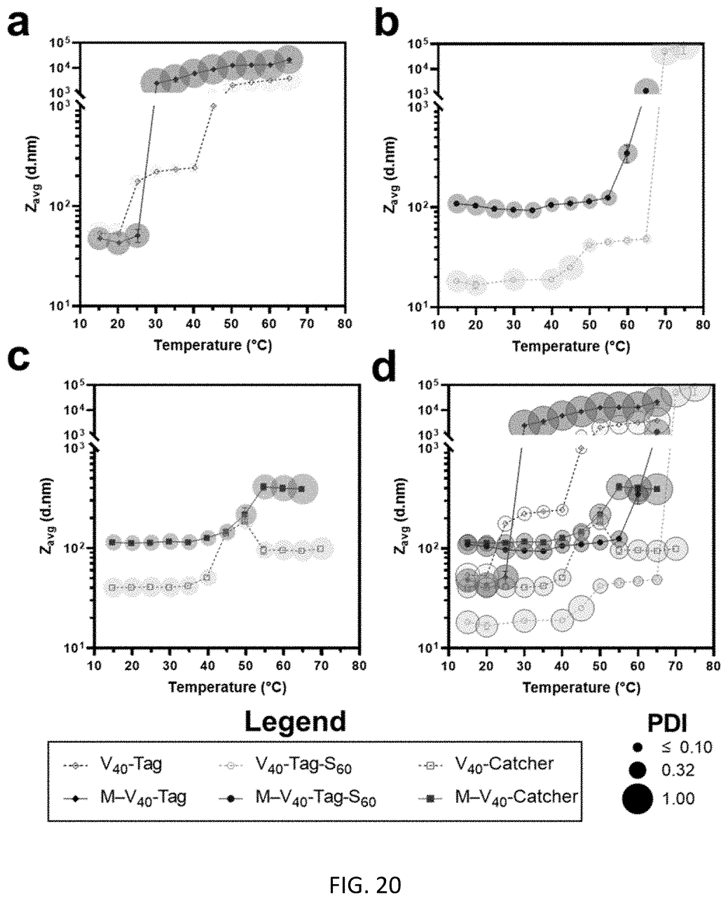

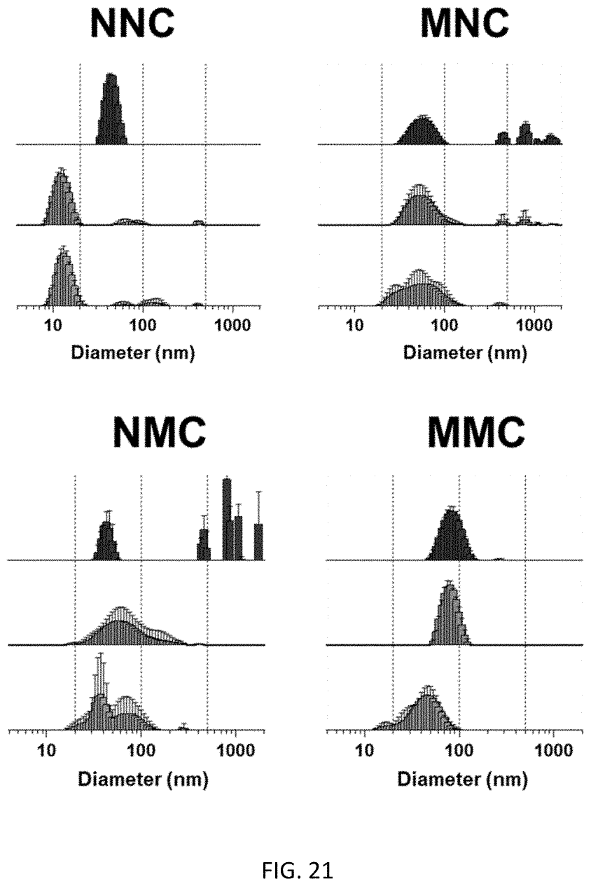

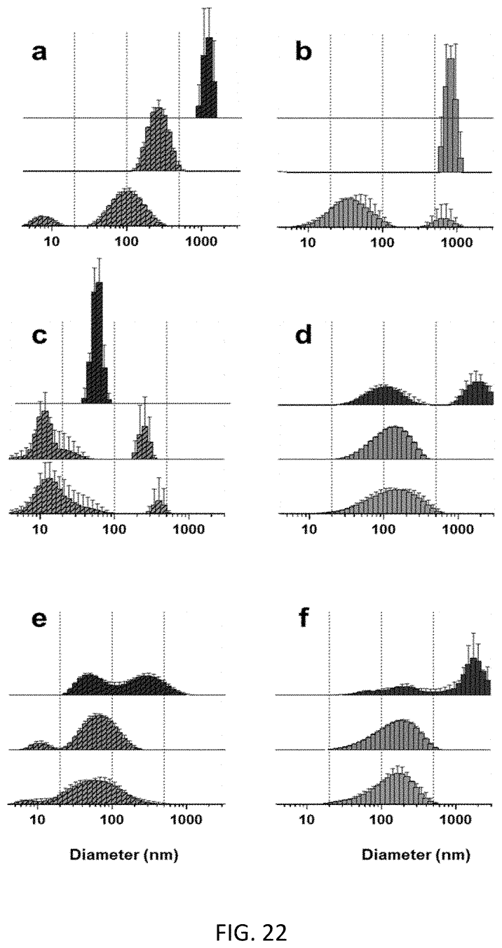

DLS. Dynamic light scattering analysis was performed on a Zetasizer Nano (Malvern Instruments, UK) with a 173° backscatter detector. Before analysis, protein solutions (20 μM in PBS) were subject to centrifugation (21,000×g, 5 min, 4° C.); supernatants were loaded into a DLS cuvette and analyzed at 15-65° C. (in 5° C. increments). Measurements were performed in triplicate at each temperature. Scattering autocorrelation functions (ACF) were analyzed with Zetasizer software using the cumulant and CONTIN methods to calculate the hydrodynamic radii (Z_{avg}), polydispersity index, and intensity-size distributions. The Zavg with PdI and intensity distributions of linear controls and star amphiphiles are shown in FIG. 20-22.

TEM. TEM imaging was performed using FEI Tecnai 12 BioTwin (ThermoFisher Scientific, Waltham, Mass.) operated at 120 kV, equipped with Gatan SC1000A CCD camera. Protein solution (10 μL) was deposited onto a carbon-coated grid. After blotting excess solution, the grid was stained with 1% uranyl acetate for 1 min and air dried at room temperature for 12 h before imaging.

Differential Interference Contrast Microscopy (DIC). DIC was conducted on a Zeiss AxioObserver Z1 widefield microscope (Carl Zeiss Inc., Berlin, Germany), with an ORCA-Flash4.0 LT+ Digital CMOS camera (Hamamatsu Photonics, Hamamatsu, Japan). Images were analyzed using MetaMorph imaging software (Molecular Devices, CA). Protein solution in PBS was heated to 60° C. and applied onto a glass slide (10 μL), shielded with a coverslip, and imaged immediately.

Molecular Dynamics Simulations. The atomistic structure of the SpyTag/SpyCatcher complex was obtained from the Protein Data Bank (PDB: 4MLI). The atomistic structures of disordered peptides (GVGVP)₄₀ and (GSGVP)₆₀ and the RS (GLYASKLFSNL) were obtained from I-TASSER (Iterative Threading ASSEmbly Refinement) server. YASARA was used to fuse the peptide arms to the SpyTag/SpyCatcher. The systems were subjected to energy minimization and equilibration steps with the input files generated from CHARMM-GUI solution builder, where the N-termini of NNC were modified by myristic acids to generate MMC, MNC, and NMC systems. The CHARMM36m force field parameters were used for disordered protein, salt (0.14 M NaCl and 0.01 M), and explicit TIP3P water. All atomistic molecular dynamics simulations were carried out using the GROMACS version 2019. Each system was energy minimized, followed by equilibration in isothermal-isochoric (NVT) and isothermal-isobaric (NPT) for 1 ns each, and production MD run under NPT conditions for 500 ns. The heavy atoms of the disordered protein were restrained during NVT and NPT equilibration. All restraints were removed during the production MD. The temperature of each system was maintained at 37° C. using the velocity-rescale thermostat with τₜ=1.0 ps. In the NPT equilibration step, isotropic pressure of 1 bar was maintained using Berendsen barostat with τₚ=5.0 ps and compressibility of 4.5×10⁻⁵ bar⁻¹. In the production MD, we used the Parrinello-Rahman barostat with τₚ=5.0 ps and compressibility of 4.5×10⁻⁵ bar⁻¹. Three-dimensional periodic boundary conditions were applied to each system. A 2 fs time step was used, and the nonbonded interaction neighbor list was updated every 20 steps. A 1.2 nm cutoff was used for the electrostatic and van der Waals interactions. The long-range electrostatic interactions were calculated using the Particle-Mesh Ewald method after a 1.2 nm cutoff. The bonds involving hydrogen atoms were constrained using the linear constraint solver (LINCS) algorithm. Besides 37° C., the MMC, NNC, MNC, and NMC systems were simulated for 200 ns at 5 and 67° C. The input structure for the additional simulations was obtained from the 37° C. production MD run. Except for temperature, other simulation parameters remained unchanged. Molecular visualization and images were rendered using PyMol, VMD, and YASARA software suites. Data analysis and plotting were performed using in-house Python scripts based on publicly hosted Python packages, such as matplotlib, scipy, and MDAnalysis.

Principal Component Analysis. The MD simulations trajectories were analyzed using in-house scripts to derive the 15 features describing aspects of form, size, and hydration of each construct in the last 200 ns of simulation. These variables include i) end-to-end distance between the three arms (F1-3); ii) the radius of gyration (R_{g}) of each arm and the branching point (S1-4); and iii) the average number of water molecules in the proximity of each domain and the average number of hydrogen bonds between each domain and surrounding water molecules (H1-8), as seen in Table 3 below.

TABLE 3
Size distributions for constructs forming worm-like micelles derived from the analysis
of TEM images.

| | T, | Length, nm | | Core, nm | | Width, nm | |
|---|---|---|---|---|---|---|---|
| Constructs | ° C. | mean ± SD (n) | | mean ± SD (n) | | mean ± SD (n) | |
| M-V₄₀-Tag | 40 | 3051 ± 11409 | (63) | n.d. | | 38 ± 10 | (61) |
| M-V₄₀-Tag-S₆₀ | 40 | 495 ± 326 | (359) | 9 ± 2 | (89) | 55 ± 11 | (87) |
| M-V₄₀-Catcher | 20 | 123 ± 85 | (216) | 7 ± 3 | (53) | 49 ± 10 | (60) |
| | 40 | 129 ± 89 | (384) | 8 ± 3 | (65) | 44 ± 9 | (84) |
| MNC | 40 | 261 ± 172 | (57) | 12 ± 3 | (54) | 77 ± 20 | (54) |
| NMC | 40 | 81 ± 28 | (77) | 22 ± 6 | (51) | 73 ± 19 | (51) |
| MMC | 20 | 125 ± 49 | (290) | 7 ± 3 | (237) | 39 ± 11 | (148) |
| | 40 | 169 ± 61 | (261) | 10 ± 3 | (224) | 33 ± 8 | (241) |

^{a}Most fibers extended beyond the imagining window. Maximum observable length measured is reported.
n.d.-not determined due to the lack of contrast between the core and corona of these structures.

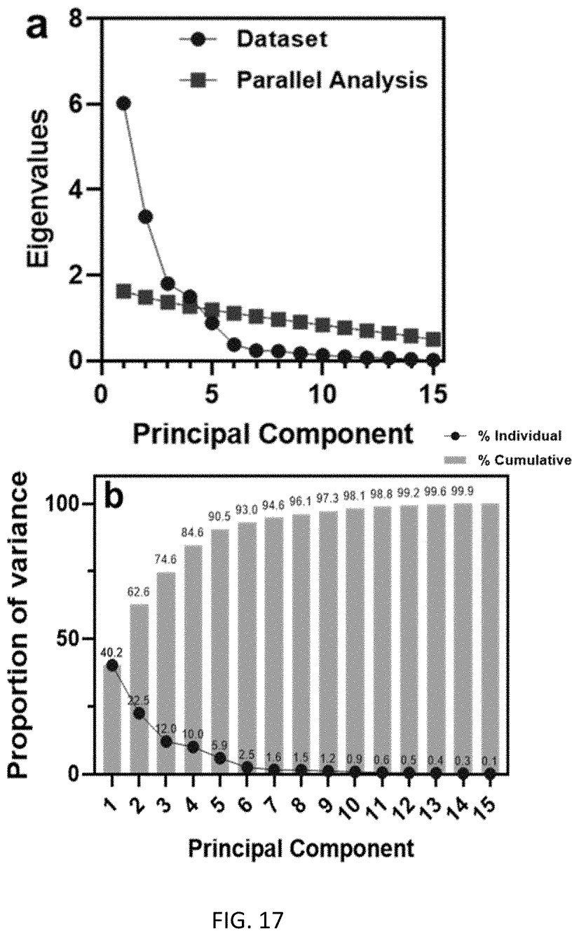

This information is used to generate a labelled dataset containing 1920 data points (15 features×4 constructs×2 temperatures×16 snapshots sampled within 170-200 ns with 2 ns intervals) as the input for PCA. First, all measurements were standardized using z-scoring (i.e., mean equal zero and standard deviation of 1) to ensure that differences in the scale and nature of these features does not bias the PCA results. The method of Horn's Parallel Analysis was used to select components with eigenvalues greater than PCs for a control dataset with identical dimension but generated “randomly” using 1000 Monte Carlo simulations) at 95 percentile (FIG. 17). The first three PCs that account for 75% of the observed variations were used for the analysis.

Statistical Analysis. Statistical analysis including PCA was performed using GraphPad Prism 9.2. The output of PCA analysis (PC and loading scores) was imported into OriginPro 2012b (version 9.8.5.204) for visualization and for calculation of 95% confidence ellipsoids in FIG. 16. The error bars for all DLS measurements represent the standard deviation of three measurements. TEM images were analyzed using ImageJ and the size, length, area distribution histograms were prepared in Prism.

What is claimed is:

1. A star miktoarm amphiphile, comprising:
a first hydrophobic arm comprised of a first repeating peptide unit having a first C-terminus and a first N-terminus;
a first hydrophilic arm comprised of a second repeating peptide unit having a second C-terminus and a second N-terminus, wherein the first hydrophilic arm is bound to the first hydrophobic arm at a junction formed by the second N-terminus and the first C-terminus; and
a second hydrophobic arm comprised of a third repeating peptide unit having a third C-terminus and a third N-terminus, wherein the second hydrophobic arm is bound by the third C-terminus to the junction of the first hydrophilic arm and the first hydrophilic arm.

2. The star miktoarm amphiphile of claim 1, wherein the first repeating peptide unit and the third repeating peptide unit are the same.

3. The star miktoarm amphiphile of claim 2, wherein the first repeating peptide unit and the third repeating peptide unit comprise GVGVP (SEQ ID NO: 1).

4. The star miktoarm amphiphile of claim 3, wherein the second repeating peptide unit comprises GSGVP (SEQ ID NO: 2).

5. The star miktoarm amphiphile of claim 4, wherein the first repeating peptide unit and the third repeating peptide unit comprise forty repeats of GVGVP (SEQ ID NO: 2).

6. The star miktoarm amphiphile of claim 5, wherein the second repeating peptide unit comprise sixty repeats of GSGVP (SEQ ID NO: 2).

7. The star miktoarm amphiphile of claim 1, wherein at least one of the first hydrophobic arm and the second hydrophobic arm are myristoylated.

8. The star miktoarm amphiphile of claim 7, wherein both the first hydrophobic arm and the second hydrophobic arm are myristoylated.

9. The star miktoarm amphiphile of claim 1, wherein the junction is formed by a first peptide fusion protein.

10. The star miktoarm amphiphile of claim 9, wherein the second hydrophobic arm is bound to a second peptide fusion protein that will irreversibly conjugate with the first peptide fusion protein.

11. A method of making a star miktoarm amphiphile, comprising the steps of:
forming a first hydrophobic arm comprises of a first repeating peptide unit having a first C-terminus and a first N-terminus;
forming a first hydrophilic arm comprised of a second repeating peptide unit having a second C-terminus and a second N-terminus
binding the first hydrophilic arm to the first hydrophobic arm at a junction formed by the second N-terminus and the first C-terminus;
forming a second hydrophobic arm comprised of a third repeating peptide unit having a third C-terminus and a third N-terminus; and
binding the second hydrophobic arm by the third C-terminus to the junction of the first hydrophilic arm and the first hydrophilic arm.

12. The method of claim 11, wherein the first repeating peptide unit and the third repeating peptide unit are the same.

13. The method of claim 12, wherein the first repeating peptide unit and the third repeating peptide unit comprise GVGVP (SEQ ID NO: 1).

14. The method of claim 13, wherein the second repeating peptide unit comprises GSGVP (SEQ ID NO: 2).

15. The method of claim 14, wherein the first repeating peptide unit and the third repeating peptide unit comprise forty repeats of GVGVP (SEQ ID NO: 1).

16. The method of claim 15, wherein the second repeating peptide unit comprise sixty repeats of GSGVP (SEQ ID NO: 2).

17. The method of claim 11, wherein at least one of the first hydrophobic arm and the second hydrophobic arm are myristoylated.

18. The method of claim 17, wherein both the first hydrophobic arm and the second hydrophobic arm are myristoylated.

19. The method of claim 11, wherein the junction is formed by a first peptide fusion protein.

20. The method of claim 19, wherein the second hydrophobic arm is bound to a second peptide fusion protein that will irreversibly conjugate with the first peptide fusion protein.


SEQUENCE LISTING (6 sequences)

SEQ ID NO: 1 - protein, 5 aa, synthetic construct
  REGION 1..5: note = polypeptide unit
GVGVP

SEQ ID NO: 2 - protein, 5 aa, synthetic construct
  REGION 1..5: note = polypeptide unit
GSGVP

SEQ ID NO: 3 - protein, 527 aa, synthetic construct
  REGION 1..527: note = fusion protein
GLYASKLFSNLGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGAHIVMVDAYKPTKGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGY

SEQ ID NO: 4 - protein, 326 aa, synthetic construct
  REGION 1..326: note = fusion protein
GLYASKLFSNLGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIG

SEQ ID NO: 5 - DNA, 61 bp, synthetic construct
  misc_feature 1..61: note = Spytag forward sequence
  source 1..61: mol_type = other DNA
cgacacatagtaatggtagacgcctacaagccgacgaagggctaatgataatgatcttcta

SEQ ID NO: 6 - DNA, 360 bp, synthetic construct
  misc_feature 1..360: note = SpyCatcher
  source 1..360: mol_type = other DNA
atgggcgttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggctaatgataatga